(19) United States
(12) Patent Application Publication
(10) Pub. No.: US 2023/0211036 A1
(43) Pub. Date: Jul. 6, 2023

(54) SYSTEM AND METHOD FOR DISINFECTING AIR IN AN AIRFLOW SYSTEM USING AN ULTRAVIOLET LASER

(71) Applicant: Pin Long, LaSalle (CA)

(72) Inventor: Pin Long, LaSalle (CA)

(21) Appl. No.: 17/566,714

(22) Filed: Dec. 31, 2021

(51) Int. Cl. A61L 9/20; F24F 8/22

(52) U.S. Cl. CPC A61L 9/20; F24F 8/22; A61L 2209/12; A61L 2209/16

(57) ABSTRACT

A system and a method for disinfecting air from airborne pathogens, for example, viruses, bacteria, etc., in an airflow system, are provided. One or more first reflectors reflect a high power, ultraviolet laser beam (UVLB) from an ultraviolet laser(s) into one or more airflow channels of the airflow system. One or more second reflectors are attached to internal surfaces of the airflow channel(s), in a free space optical connection to the first reflector(s), for reflecting the UVLB and increasing contact, utilization, and interaction of the UVLB with the airflow in the airflow channel(s) for irradiating and disinfecting the air therein. Air supply components on a ceiling blow the disinfected air from an airflow channel into an enclosed space for inhalation by occupants in the enclosed space, while air exhaust components on a floor exhaust air exhaled by the occupants into an airflow channel, thereby precluding cross-contamination in the enclosed space.

### BACKGROUND

Pathogens are infectious microorganisms comprising, for example, viruses, bacteria, protozoans, fungi or molds, and other disease-causing microorganisms. Pathogens cause diseases and/or elicit serious health issues when they enter human and animal bodies. For example, humans may get exposed to harmful, airborne pathogens in a circulating airflow system, for example, an air conditioning system, in a local area or a room of a residential home, a residential building, an apartment building, an office building, a hospital, an airport, a commercial and business establishment such as a restaurant, a shopping mall, etc., in a transport vehicle such as a subway train, a bus, etc. Cross-contamination of air by occupants in a local area, or a room, or a transport vehicle pose a risk of transmission of pathogens that spread diseases through an entire community, especially during the course of an epidemic or a pandemic. One approach to prevent or minimize the transmission of these pathogens is by disinfecting air in an airflow system, that is circulating and/or flows past human beings. As used herein, “disinfecting air in an airflow system” comprises inactivation and/or killing of airborne pathogens comprising, for example, viruses, bacteria, protozoans, fungi or molds, disease-causing microorganisms, etc., in the airflow system.

Studies have shown that pathogens can be inactivated or killed by ultraviolet (UV) irradiation that results in an irrevocable chemical change in the deoxyribonucleic acid (DNA) of the pathogens and renders the pathogens inactive, thereby destroying their ability to reproduce and multiply. Ultraviolet light sources, for example, ultraviolet-C (UVC) lamps or UVC light emitting diodes (LEDs) produce UVC radiation at particular wavelengths that are used for inactivating or killing pathogens. However, these UVC lamps or UVC LEDs produce continuous, ultraviolet light at low energy and low output power or low light intensity, which takes a substantial amount of time to inactivate or kill the airborne pathogens in a large volumetric airflow rate. Moreover, UVC lamps or UVC LEDs do not allow sufficient exposure of the airflow to the ultraviolet radiation produced therefrom, and therefore the airflow does not receive a sufficient dosage of the ultraviolet radiation to facilitate an irreversible chemical reaction to the DNA of each pathogen in the airflow and disinfection of the airflow.

Furthermore, in many conventional airflow systems, for example, air conditioning systems, air inlet channels and air outlet channels are located in or near each other at the same level in an enclosed space. Even when output air from these conventional air conditioning systems is disinfected, the air in the enclosed space gets quickly infected and/or contaminated by pathogens because air exhaled by occupants in the enclosed space is not directly exhausted to an external environment, thereby resulting in the infected and/or contaminated air being inhaled by other occupants in the enclosed space.

Hence, there is a long-felt need for a system and a method for irradiating and disinfecting air in an airflow system in a short time, using one or more ultraviolet lasers. Furthermore, there is a need for a system and a method for minimizing and/or precluding cross-contamination of air in an enclosed space.

### SUMMARY

This summary is provided to introduce a selection of concepts in a simplified form that are further disclosed in the detailed description. This summary is not intended to determine the scope of the claimed subject matter.

The system and the method disclosed herein address the above-recited need for irradiating and disinfecting air in an airflow system in a short time, using one or more ultraviolet lasers. Furthermore, the system and the method disclosed herein address the above-recited need for minimizing and/or precluding cross-contamination of air in an enclosed space. The airflow system is, for example, an air conditioning system, an air exchange system, an air purifier system, an air circulation system, etc. The system disclosed herein comprises one or more ultraviolet lasers and multiple reflectors. The ultraviolet laser(s) is configured to generate and direct a high power, ultraviolet laser beam into the airflow system for inactivating and/or killing pathogens contained in the air flowing through the airflow system. In an embodiment, the ultraviolet laser(s) is a pulsed ultraviolet laser. The wavelength of pulses of the high power, ultraviolet laser beam generated by the pulsed ultraviolet laser is in a range, for example, from about 150 nanometers (nm) to about 300 nanometers. A pulse duration of the high power, ultraviolet laser beam generated by the ultraviolet laser(s) is, for example, in one of milliseconds, microseconds, nanoseconds, picoseconds, and femtoseconds. In another embodiment, the ultraviolet laser(s) is a continuous wave ultraviolet laser. The wavelength of a continuous wave of the high power, ultraviolet laser beam generated by the continuous wave ultraviolet laser is in a range, for example, from about 150 nm to about 300 nm.

One or more first reflectors are positioned in a free space optical connection to the ultraviolet laser(s). The first reflector(s) is configured and positioned to reflect and direct the ultraviolet laser beam into one or more airflow channels of the airflow system. The airflow channels comprise, for example, an air inlet channel configured to allow entry of the air into the airflow system, and an air outlet channel configured to exhaust the air from the airflow system. One or more second reflectors are positioned in a free space optical connection to the first reflector(s). That is, one or more second reflectors are positioned in an optical path of the high power, ultraviolet laser beam directed by the first reflector(s) to reflect the high power, ultraviolet laser beam within and throughout the length of the airflow channel(s). The second reflectors are attached to internal surfaces of the airflow channel(s) of the airflow system to allow the high power, ultraviolet laser beam to be reflected within and throughout the length of the airflow channel(s). In an embodiment, the system further comprises a light transmission window positioned at a predetermined location on the airflow channel(s) of the airflow system. The light transmission window is configured to pass and direct the high power, ultraviolet laser beam generated by the ultraviolet laser(s) and reflected by the first reflector(s) to the second reflectors in the airflow channel(s) of the airflow system. The first reflector(s) reflects the high power, ultraviolet laser beam into the light transmission window positioned on an internal surface of the airflow channel(s). The first reflector(s) reflects the high power, ultraviolet laser beam into the airflow channel(s) via the light transmission window. The second reflectors are configured to reflect the reflected ultraviolet laser beam multiple times within and throughout the length of the airflow channel(s) in multiple directions, with the reflected ultraviolet laser beam forming an ultraviolet laser beam tunnel to increase contact, utilization, and interaction of the reflected ultraviolet laser beam with the air flowing in the airflow channel(s) of the airflow system, for irradiating and disinfecting the air flowing through the airflow channel(s). The second reflectors located inside the airflow channel(s) reflect the high power, ultraviolet laser beam in multiple directions, for example, in a backward direction, a forward direction, an upward direction, a downward direction, etc., to form the ultraviolet laser beam tunnel to maximize the interaction between the high power, ultraviolet laser beam and the airflow. In an embodiment, the system further comprises one or more third reflectors positioned in a free space optical connection to the first reflector(s) and the second reflectors. The third reflector(s) is operably coupled to one or more of the internal surfaces of the airflow channel(s) of the airflow system and optically aligned with the second reflectors in the airflow channel(s) of the airflow system. The third reflector(s) is configured to reflect and direct the high power, ultraviolet laser beam reflected by the first reflector(s) to the second reflectors to further increase the contact, the utilization, and the interaction of the reflected ultraviolet laser beam with the air flowing in the airflow channel(s) of the airflow system.

Disclosed herein is also a system for disinfecting air in an airflow system and for minimizing and/or precluding cross-contamination of the air in an enclosed space, for example, a room of a residential home, a residential building, an apartment building, an office building, a hospital, an airport, a commercial and business establishment such as a restaurant, a shopping mall, etc., in a transport vehicle such as a subway train, a bus, etc. The system comprises an airflow system, multiple air supply components, multiple air exhaust components, and a disinfection system. The airflow system is configured to allow a flow of air through airflow channels. The airflow channels comprise an air inlet channel and an air outlet channel. The air inlet channel extends below a floor of the enclosed space. The air outlet channel extends above a ceiling of the enclosed space. The air supply components are operably coupled to and in fluid communication with the air outlet channel of the airflow system. In an embodiment, the air supply components are air blowers or fans configured to blow the disinfected air into the enclosed space. In another embodiment, the air supply components are ports or openings configured to direct the disinfected air into the enclosed space. The air supply components extend into the enclosed space from the ceiling of the enclosed space. The air exhaust components are operably coupled to and in fluid communication with the air inlet channel. In an embodiment, the air exhaust components are air suction devices configured to suction the air exhaled by occupants of the enclosed space and exhaust the exhaled air into the air inlet channel of the airflow system. In another embodiment, the air exhaust components are configured as ports or openings for exhausting the exhaled air into the air inlet channel of the airflow system. The air exhaust components are positioned inside the enclosed space on the floor of the enclosed space. The air exhaust components are configured to receive air exhaled by occupants in the enclosed space and exhaust the exhaled air into the air inlet channel of the airflow system for subsequent irradiation and disinfection by the high power, ultraviolet laser beam generated by the ultraviolet laser(s) positioned proximal to one or both of the airflow channels of the airflow system. The disinfection system is in operable communication with one or more of the airflow channels, for example, the air inlet channel and/or the air outlet channel of the airflow system. The disinfection system comprises one or more ultraviolet lasers and multiple reflectors as disclosed above. The air supply components blow the irradiated, disinfected air output from the disinfection system, from the air outlet channel of the airflow system, into the enclosed space for inhalation by the occupants in the enclosed space, while the air exhaust components receive and exhaust the air exhaled by the occupants in the enclosed space into the air inlet channel of the airflow system for subsequent irradiation and disinfection by the high power, ultraviolet laser beam generated by the ultraviolet laser(s) positioned proximal to one or both of the air inlet channel and the air outlet channel of the airflow system, thereby minimizing and/or precluding cross-contamination of the air in the enclosed space.

In an embodiment, the system for disinfecting air in an airflow system and for minimizing and/or precluding cross-contamination of the air in an enclosed space comprises guide members extending upwardly into the enclosed space from the floor of the enclosed space. The guide members are positioned between each of the air supply components and a corresponding one of the air exhaust components. In an embodiment, the guide members are configured, for example, as separators, to separate the enclosed space into separate enclosed spaces, where the air flows from the air supply components into respective enclosed spaces and air flows out of the enclosed spaces through their respective air exhaust components. The guide members are configured to allow only a vertical flow of the air within the enclosed space, from the ceiling to the floor of the enclosed space, and preclude a horizontal flow of the air within the enclosed space. The guide members are configured to guide the irradiated and disinfected air blown by the air supply components to the occupants in the enclosed space for inhalation by the occupants in the enclosed space and to guide the air exhaled by the occupants in the enclosed space into the air exhaust components, thereby minimizing and/or precluding cross-contamination of the air in the enclosed space.

Disclosed herein are also methods for disinfecting air in an airflow system and for minimizing and/or precluding cross-contamination of the air in an enclosed space using the systems disclosed above. In one or more embodiments, related systems comprise circuitry for executing the methods disclosed herein. The circuitry is of any combination of hardware, software, and/or firmware configured to execute the methods disclosed herein depending upon the design choices of a system designer. In an embodiment, various structural elements are employed depending on the design choices of the system designer.

### BRIEF DESCRIPTION OF THE DRAWINGS

The foregoing summary, as well as the following detailed description, is better understood when read in conjunction with the appended drawings. For illustrating the embodiments herein, exemplary constructions of the embodiments are shown in the drawings. However, the embodiments herein are not limited to the specific components and methods disclosed herein. The description of a component or a method step referenced by a numeral in a drawing is applicable to the description of that component or method step shown by that same numeral in any subsequent drawing herein.

FIG. 1A exemplarily illustrates an embodiment of a disinfection system positioned in communication with an air outlet channel for disinfecting air flowing in the air outlet channel.

FIG. 1B exemplarily illustrates a cross-sectional view of the air outlet channel taken along a section A-A shown in FIG. 1A.

FIG. 2 exemplarily illustrates an embodiment of the disinfection system positioned in communication with an air outlet channel of an airflow system for disinfecting air flowing in the air outlet channel.

FIG. 3 exemplarily illustrates an embodiment of the disinfection system comprising an additional reflector positioned in communication with an air outlet channel for disinfecting air flowing in the air outlet channel.

FIG. 4A exemplarily illustrates an embodiment of the disinfection system positioned in communication with an air inlet channel of an airflow system for disinfecting air flowing in the air inlet channel.

FIG. 4B exemplarily illustrates a cross-sectional view of the air inlet channel taken along a section B-B shown in FIG. 4A.

FIG. 5 exemplarily illustrates an embodiment of the disinfection system comprising an additional reflector positioned in communication with an air inlet channel for disinfecting air flowing in the air inlet channel.

FIG. 6 exemplarily illustrates an embodiment of the disinfection system positioned in communication with an air inlet channel and an air outlet channel of an airflow system for disinfecting air flowing in the air inlet channel and the air outlet channel.

FIG. 7 illustrates a flowchart of an embodiment of a method for disinfecting air in an airflow system.

FIG. 8 exemplarily illustrates an embodiment of a system for disinfecting air and for minimizing and/or precluding cross-contamination of the air in an enclosed space.

FIG. 9A exemplarily illustrates a side view of an embodiment of a system for disinfecting air and for minimizing and/or precluding cross-contamination of the air in an enclosed space.

FIG. 9B exemplarily illustrates a top view of a ceiling of the enclosed space shown in FIG. 9A, in the embodiment of the system for disinfecting air and for minimizing and/or precluding cross-contamination of the air in the enclosed space.

FIG. 9C exemplarily illustrates a cutaway view, showing a top view of a floor of the enclosed space shown in FIG. 9A, in the embodiment of the system for disinfecting air and for minimizing and/or precluding cross-contamination of the air in the enclosed space.

FIG. 10 illustrates a flowchart of an embodiment of a method for disinfecting air and for minimizing and/or precluding cross-contamination of the air in an enclosed space.

### DETAILED DESCRIPTION

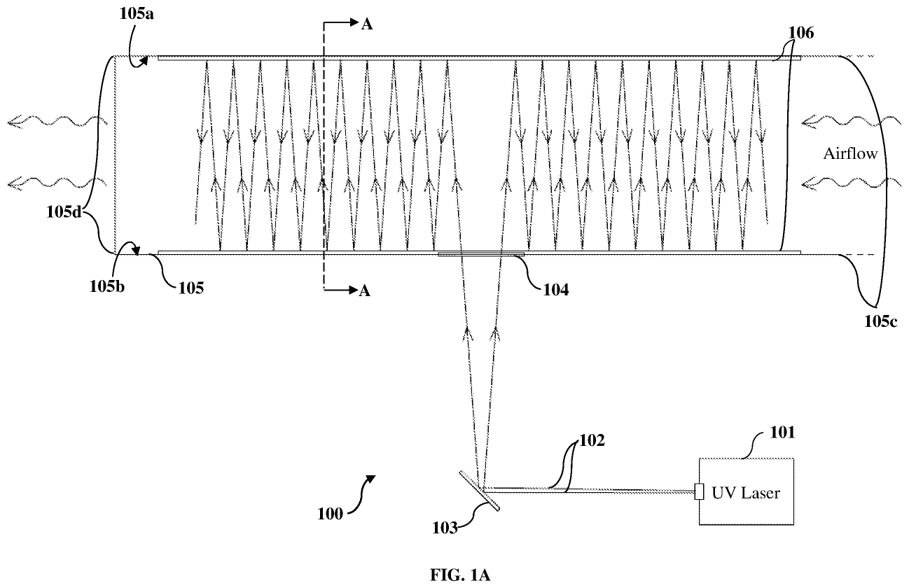

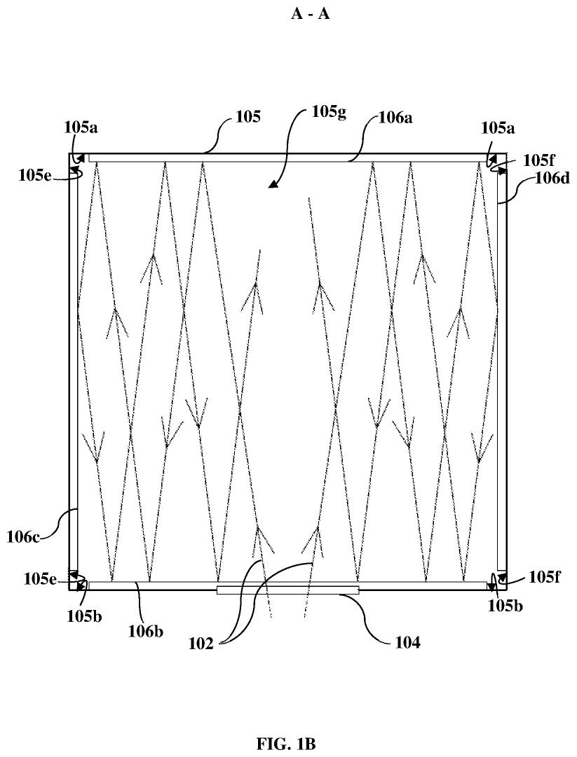

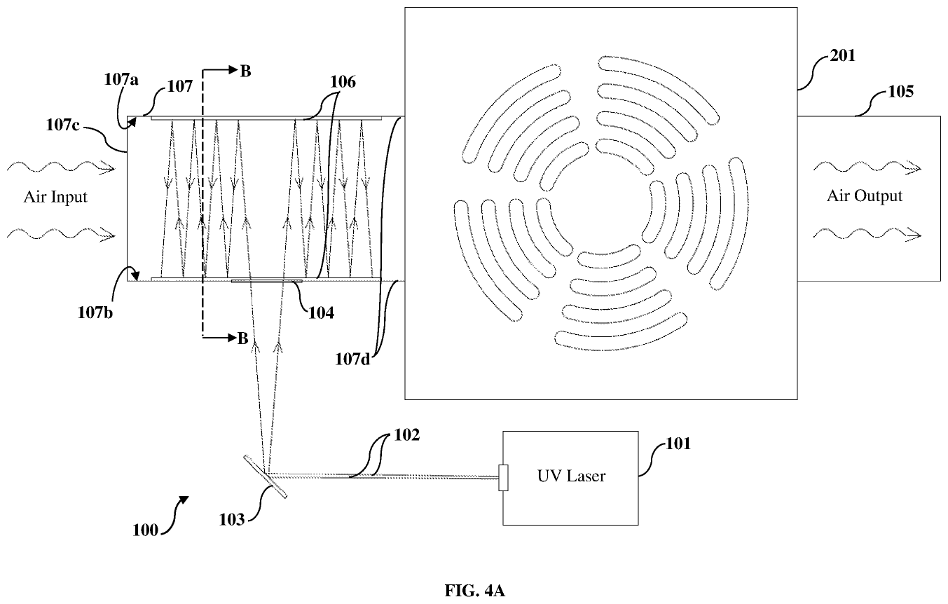

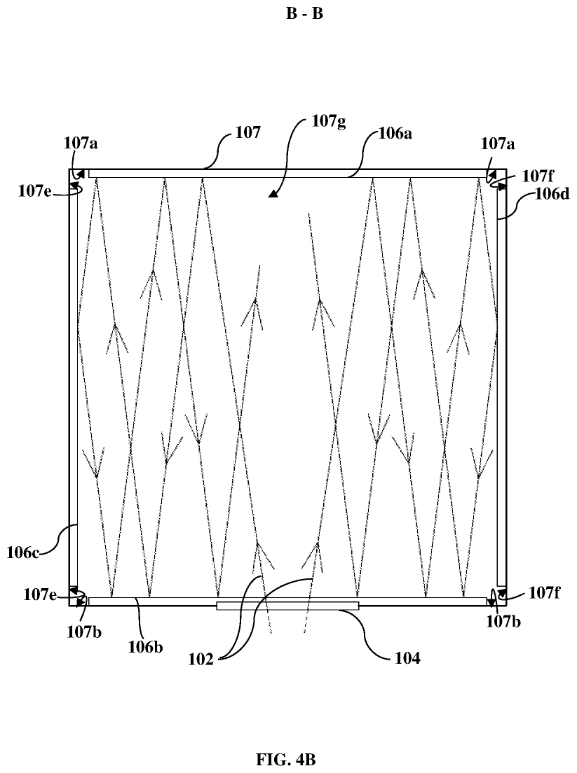

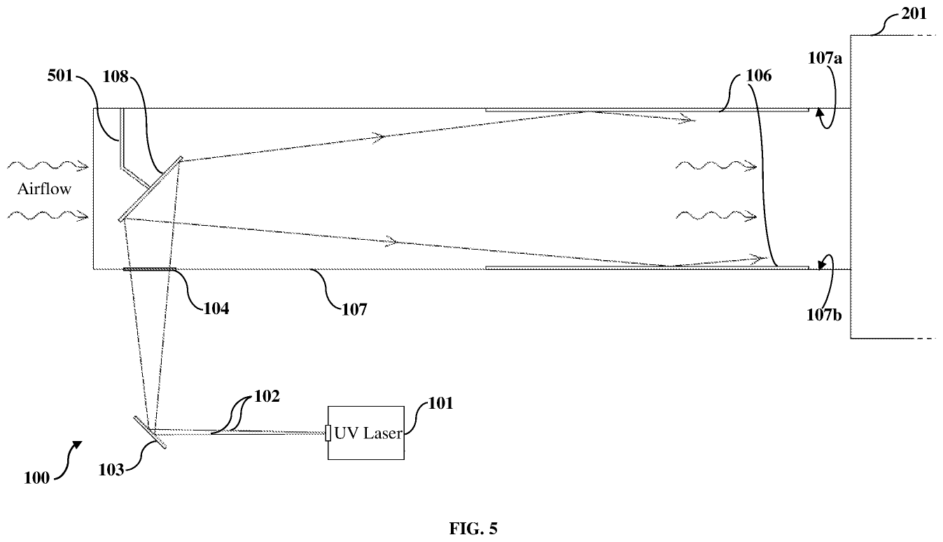

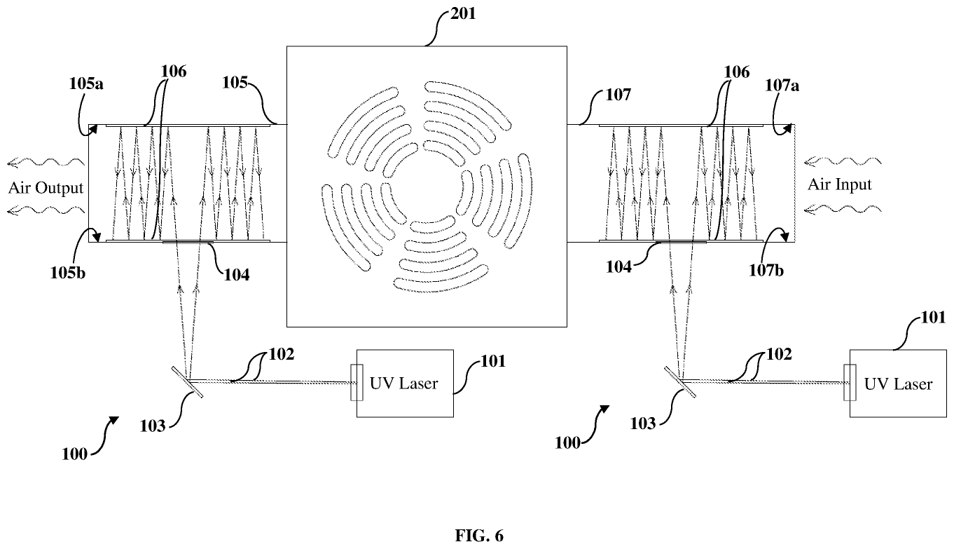

FIG. 1A exemplarily illustrates an embodiment of a disinfection system 100 positioned in communication with an air outlet channel 105 for disinfecting air flowing in the air outlet channel 105. For purposes of illustration, the disclosure herein refers to a disinfection system 100 being positioned at one airflow channel, for example, the air outlet channel 105 of an airflow system 201 as exemplarily illustrated in FIG. 1A and FIGS. 2-3 for disinfecting the air flowing through the air outlet channel 105; however, the scope of the system and the methods disclosed herein is not limited to the disinfection system 100 being positioned at the air outlet channel 105 of the airflow system 201, but may be extended to being positioned alternatively at an air inlet channel 107 of the airflow system 201 as exemplarily illustrated in FIG. 4A and FIG. 5 for disinfecting the air flowing through the air inlet channel 107, or at both the air outlet channel 105 and the air inlet channel 107 of the airflow system 201 as exemplarily illustrated in FIG. 6 for disinfecting the air flowing through both the air outlet channel 105 and the air inlet channel 107. The disinfection system 100 irradiates and disinfects air flowing through an airflow channel, for example, the air outlet channel 105 exemplarily illustrated in FIG. 1A, of any large volume, airflow system using one or more ultraviolet lasers 101. The air outlet channel 105 is configured to exhaust air from the airflow system 201. As exemplarily illustrated in FIG. 1A, the disinfection system 100 is positioned in communication with the air outlet channel 105 through which air, that may contain pathogens, for example, viruses, bacteria, protozoans, fungi or molds, any disease-causing microorganisms, etc., flows from one end 105c to another end 105d of the air outlet channel 105.

As exemplarily illustrated in FIG. 1A, the disinfection system 100 comprises an ultraviolet laser 101 and multiple reflectors 103 and 106. The ultraviolet laser 101 is configured to generate and direct a high power, ultraviolet laser beam 102 into the air outlet channel 105 for inactivating and/or killing the pathogens contained in the air flowing through the air outlet channel 105. The average power of the ultraviolet laser 101 ranges, for example, from several hundreds of mini watts to several hundreds of watts. In an embodiment, the ultraviolet laser 101 is a pulsed ultraviolet laser. Examples of the pulsed ultraviolet laser utilized in the disinfection system 100 comprise a pulsed ultraviolet fiber laser, a pulsed ultraviolet solid-state laser, an excimer laser, etc. The peak power of the pulsed ultraviolet laser ranges from few watts (W) to several hundred megawatts (MW), several hundred gigawatts (GW), and several hundred terawatts (TW) in various embodiments. For example, the peak power of the pulsed ultraviolet laser is about 10 watts to about 100 terawatts. The repetition rate of the pulsed ultraviolet laser ranges, for example, from few hertz (Hz) to several hundred megahertz (MHz). For example, the repetition rate of the pulsed ultraviolet laser is about 1 Hz to about 500 MHz. The wavelength of pulses of the high power, ultraviolet laser beam 102 generated by the pulsed ultraviolet laser is in a range, for example, from about 150 nanometers (nm) to about 300 nanometers. In an embodiment, the wavelength of the ultraviolet laser beam 102 is, for example, from about 150 nm to about 390 nm.

The number of pulses of the high power, ultraviolet laser beam 102 provided by the ultraviolet laser 101 for irradiating the airflow is, for example, about 1 pulse to about 100 million pulses. The peak power of the pulses of the high power, ultraviolet laser beam 102 provided by the ultraviolet laser 101 is, for example, about 10 milliwatts (mW) to about 100 kilowatts (kW). A pulse duration of the high power, ultraviolet laser beam 102 generated by the ultraviolet laser 101 ranges, for example, from milliseconds, microseconds, nanoseconds, and picoseconds to femtoseconds in various embodiments. In an embodiment, the pulse duration of the high power, ultraviolet laser beam 102 is in a range of about 1 millisecond to about 999 milliseconds. In another embodiment, the pulse duration of the high power, ultraviolet laser beam 102 is in a range of about 1 microsecond to about 999 microseconds. In another embodiment, the pulse duration of the high power, ultraviolet laser beam 102 is in a range of about 1 nanosecond to about 999 nanoseconds. In another embodiment, the pulse duration of the high power, ultraviolet laser beam 102 is in a range of about 1 picosecond to about 999 picoseconds. In another embodiment, the pulse duration of the high power, ultraviolet laser beam 102 is in a range of about 1 femtosecond to about 999 femtoseconds.

With the same energy but a short time duration, for example, in a picosecond-range, and in an embodiment in a femtosecond range, the peak power of the pulsed ultraviolet laser is substantially high, for example, in a megawatt-range, and in an embodiment in a gigawatt-range, thereby causing the pulsed ultraviolet laser beam to disinfect the air in the airflow system 201. For example, a pulsed ultraviolet laser with a 1-watt average output power, a 1 kilohertz (kHz)-repetition rate, and a 100-femtosecond pulse duration, produces a peak power of about 9.4 gigawatts to disinfect the air in the airflow system 201 and inactivate the airborne pathogens therein. In another embodiment, the ultraviolet laser 101 is a continuous wave ultraviolet laser. Examples of the continuous wave ultraviolet laser comprise a continuous wave ultraviolet fiber laser, a continuous wave ultraviolet solid-state laser, etc. The wavelength of a continuous wave of the high power, ultraviolet laser beam 102 generated by the continuous wave ultraviolet laser is in a range, for example, from about 150 nm to about 300 nm. In an embodiment, the ultraviolet laser 101 is externally positioned proximal to the air outlet channel 105 as exemplarily illustrated in FIG. 1A. For purposes of illustration, the disclosure herein refers to a single ultraviolet laser 101 being used in the disinfection system 100; however, the scope of the system and the method disclosed herein is not limited to the use of a single ultraviolet laser 101, but may be extended to include more than one ultraviolet laser 101 to direct one or more high power, ultraviolet laser beams 102 into the airflow channel(s) 105 and/or 107.

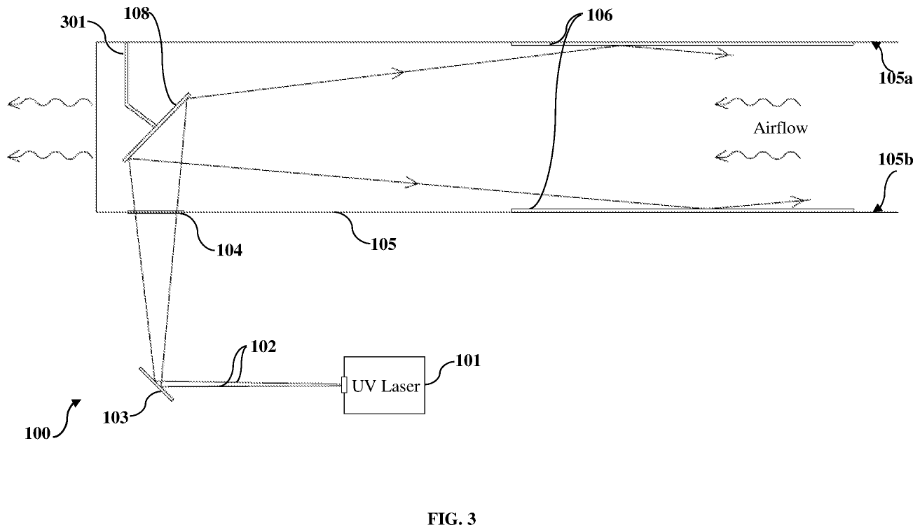

One or more first reflectors, for example, 103, are positioned in a free space optical connection to the ultraviolet laser 101. For example, a first reflector 103, for example, a reflective mirror, is positioned in a free space optical connection to the ultraviolet laser 101 as exemplarily illustrated in FIG. 1A. As used herein, “free space optical connection” refers to an optical relationship between two optical elements, for example, between the ultraviolet laser 101 and the reflector 103, and between the reflectors 103, 106, and 108 exemplarily illustrated in FIG. 3, that allows the optical elements to receive, reflect, pass, redirect, and transfer light, for example, the ultraviolet laser beam 102, through free space, for example, through air, a vacuum, an enclosed space, an airflow channel 105 or 107, etc., therebetween. The free space optical connection is an optical communication technique for communicating the ultraviolet laser beam 102 through free space between the optical elements. The free space acts as a communication medium between the optical elements for propagating the ultraviolet laser beam 102 therebetween. To establish a free space optical connection, the optical elements are configured to be in line-of-sight of each other to create a beam transmission path in free space to propagate the ultraviolet laser beam 102 in free space. The first reflector 103 is of a geometrical shape, for example, a circular shape, a square shape, a rectangular shape, etc., with a length ranging, for example, from about 5 millimeters (mm) to about 1000 mm. The thickness of the first reflector 103 is, for example, from about 1 mm to about 100 mm. The ultraviolet laser 101 directs the high power, ultraviolet laser beam 102 to the first reflector 103. The first reflector 103 is configured and positioned to reflect and direct the ultraviolet laser beam 102 into one or more airflow channels 105 and/or 107 of the airflow system 201. In an embodiment, more than one first reflector 103 is configured to be positioned in a free space optical connection to the ultraviolet laser 101 for reflecting and directing the ultraviolet laser beam 102 into one or more airflow channels 105 and/or 107 of the airflow system 201.

In an embodiment, the first reflector 103 is positioned proximal to the air outlet channel 105 as exemplarily illustrated in FIG. 1A. The first reflector 103 reflects the high power, ultraviolet laser beam 102 directed by the ultraviolet laser 101 into the air outlet channel 105. One or more second reflectors 106 are attached to internal surfaces 105a, 105b, etc., of the air outlet channel 105. For example, four second reflectors 106a, 106b, 106c, and 106d are positioned on the internal surfaces 105a, 105b, 105e, and 105f of the air outlet channel 105 respectively, as exemplarily illustrated in FIG. 1B. The second reflectors 106 are, for example, reflective mirrors, reflective mirror films, thin reflective films, reflective mirror plates, etc., attached to the internal surfaces 105a, 105b, etc., of the air outlet channel 105, for example, using adhesive materials, fasteners, etc. In an embodiment, the second reflectors 106 are made, for example, from glass plates with aluminum coatings, silver coatings, etc. In another embodiment, the second reflectors 106 are mirror films made, for example, of plastic sheets coated with aluminum or other metal coatings. In another example, the second reflectors 106 are made from polished metal plates. In an embodiment, the second reflectors 106 are configured to be positioned on other internal surfaces and at the ends of the air outlet channel 105 for reflecting the high power, ultraviolet laser beam 102 within the air outlet channel 105 multiple times. In another embodiment, the second reflectors 106 are positioned inside the surfaces of the airflow channel(s) 105 and/or 107. In an embodiment, the second reflectors 106 are of geometrical shapes, for example, a circular shape, a square shape, a rectangular shape, etc., with lengths ranging, for example, from about 100 mm to about 10000 mm. The thicknesses of the second reflectors 106 are, for example, from about 1 mm to about 100 mm. In an embodiment, the second reflectors 106 cover the entire internal surface area of the airflow channel(s) 105 and/or 107.

In an embodiment, the disinfection system 100 further comprises a light transmission window 104 positioned at a predetermined location on the air outlet channel 105. For example, the light transmission window 104 is centrally positioned, proximal to the internal surface 105b of the air outlet channel 105 as exemplarily illustrated in FIG. 1A. The light transmission window 104 is attached to the internal surface 105b of the air outlet channel 105, for example, using adhesive materials, fasteners, etc. In an example, the light transmission window 104 is made of glass configured to optimally transmit the high power, ultraviolet laser beam 102 reflected by the first reflector 103 into the air outlet channel 105. The shape of the light transmission window 104 is, for example, a circular shape, a rectangular shape, etc. In an example, the diameter of the circular shaped-light transmission window 104 is about 5 mm to 500 mm, with a thickness from about 1 mm to about 50 mm. The width and the length of the rectangular shaped-light transmission window 104 are, for example, from about 5 mm to about 1000 mm. The second reflectors 106 are positioned in a free space optical connection to the first reflector 103 via the light transmission window 104. The second reflectors 106 are installed in optical alignment with the light transmission window 104 to fully utilize the incident high power, ultraviolet laser beam 102 and maximize interaction of the high power, ultraviolet laser beam 102 with the air flowing through the air outlet channel 105 to disinfect the air. The first reflector 103 is optically aligned with the centrally positioned light transmission window 104 as exemplarily illustrated in FIG. 1A. Optical alignment refers to an arrangement of two or more optical elements, for example, the first reflector 103 and the light transmission window 104, in a free space optical connection to each other, allowing for transmission or transfer of a majority of light between them. The light transmission window 104 is configured to pass, direct, and transmit the high power, ultraviolet laser beam 102 generated by the ultraviolet laser 101 and reflected by the first reflector 103 to the second reflectors 106 in the air outlet channel 105.

The second reflectors 106 are configured to reflect the reflected ultraviolet laser beam 102 multiple times within and throughout the length of the air outlet channel 105 in multiple directions, for example, a backward direction, a forward direction, an upward direction, a downward direction, etc., with the reflected ultraviolet laser beam 102 forming an ultraviolet laser beam tunnel to increase contact, utilization, and interaction of the reflected ultraviolet laser beam 102 with the air flowing in the air outlet channel 105 for irradiating and disinfecting the air in a short time. The high power, ultraviolet laser beam 102 reflected by the first reflector 103 and passing through the light transmission window 104 into the air outlet channel 105 is reflected multiple times between the second reflectors 106. The multi-reflecting high power, ultraviolet laser beam 102 within the air outlet channel 105 exposes the air flowing through the air outlet channel 105 to high power, ultraviolet radiation, for example, ultraviolet-C (UVC) radiation of wavelength in a range of, for example, about 150 nm to about 300 nm, thereby irradiating and disinfecting the air in a short time. The irradiation of the air flowing through the air outlet channel 105 by the multi-reflecting high power, ultraviolet laser beam 102 inactivates and/or kills the pathogens in the airflow. Because of the high peak power of the ultraviolet laser beam 102, the time for disinfecting the air flowing through the air outlet channel 105 is reduced, for example, from several minutes to several seconds. In an example, the time range for exposing the airflow in the air outlet channel 105 to the ultraviolet laser beam 102 for irradiating and disinfecting the air is about 1 second to about 100 minutes. Depending on the actual air flow volume and the output power of the ultraviolet laser 101, the time for irradiating and disinfecting the air in the air outlet channel 105 is, for example, about 1 second to about 30 minutes.

FIG. 1B exemplarily illustrates a cross-sectional view of the air outlet channel 105 taken along a section A-A shown in FIG. 1A. In an embodiment, the second reflectors 106a, 106b, 106c, and 106d are positioned inside the surfaces 105a, 105b, 105e, and 105f of the air outlet channel 105 respectively. The cross-sectional view in FIG. 1B exemplarily illustrates four second reflectors 106a, 106b, 106c, and 106d attached to the internal surfaces 105a, 105b, 105e, and 105f of the air outlet channel 105 respectively. In another embodiment, the second reflectors 106a, 106b, 106c, and 106d cover the entire internal surface area of the air outlet channel 105. In an embodiment, the first reflector 103 exemplarily illustrated in FIG. 1A, reflects the incoming high power, ultraviolet laser beam 102 and redirects the divergent, high power, ultraviolet laser beam 102 into the air outlet channel 105 via the light transmission window 104. The second reflectors 106a, 106b, 106c, and 106d reflect the divergent, high power, ultraviolet laser beam 102 multiple times in multiple directions, for example, opposing directions such as upward and downward directions, back and forth directions, etc., from the internal surfaces 105a, 105e to other internal surface 105b, 105f of the air outlet channel 105 as exemplarily illustrated in FIG. 1B. In an example, the second reflectors 106a and 106b reflect the high power, ultraviolet laser beam 102 vertically in upward and downward directions from one internal surface 105a to another internal surface 105b of the air outlet channel 105 and vice versa. Similarly, the second reflectors 106c and 106d reflect the high power, ultraviolet laser beam 102 horizontally in back and forth directions from one internal surface 105e to another internal surface 105f of the air outlet channel 105 and vice versa. The second reflectors 106a, 106b, 106c, and 106d, therefore, reflect the high power, ultraviolet laser beam 102 all around the inside of the air outlet channel 105, thereby forming an ultraviolet laser beam tunnel to increase contact of the high power, ultraviolet laser beam 102 with the air flowing through the air outlet channel 105 and to maximize the interaction between the air flowing through the air outlet channel 105 and the high power, ultraviolet laser beam 102.

As exemplarily illustrated in FIG. 1B, the ultraviolet laser beam 102 at the entrance of the light transmission window 104 is directed to the reflector 106a on the upper internal surface 105a of the air outlet channel 105 in opposing directions, from where the ultraviolet laser beam 102 incident on the reflector 106a is reflected in the general direction of the reflector 106b on the lower internal surface 105b of the air outlet channel 105. This reflection of the ultraviolet laser beam 102 proceeds in opposing directions to the reflectors 106c and 106d on the internal surfaces 105e and 105f of the air outlet channel 105 respectively, from where the ultraviolet laser beam 102 is reflected between the reflectors 106b and 106a multiple times. The ultraviolet laser beam 102 reflected from the reflector 106a located on the upper internal surface 105a of the air outlet channel 105 to the reflector 106b located on the lower internal surface 105b and back to the reflector 106a on the upper internal surface 105a of the air outlet channel 105, permeates the entire space 105g within the air outlet channel 105 and contacts the air flowing through the air outlet channel 105. The ultraviolet laser beam 102 in the air outlet channel 105 inactivates the pathogens in the air flowing through the air outlet channel 105.

The reflection of the divergent high power, ultraviolet laser beam 102 multiple times in multiple directions from internal surfaces 105a, 105e to the other internal surfaces 105b, 105f of the air outlet channel 105 provides maximum direct exposure of the air flowing through the air outlet channel 105 to the multi-reflecting high power, ultraviolet laser beam 102, thereby allowing the air to receive a substantial dosage of the ultraviolet radiation, for example, the ultraviolet-C (UVC) radiation, to facilitate an irreversible chemical reaction to the deoxyribonucleic acid (DNA) of each pathogen in the airflow through the air outlet channel 105 and disinfection of the airflow.

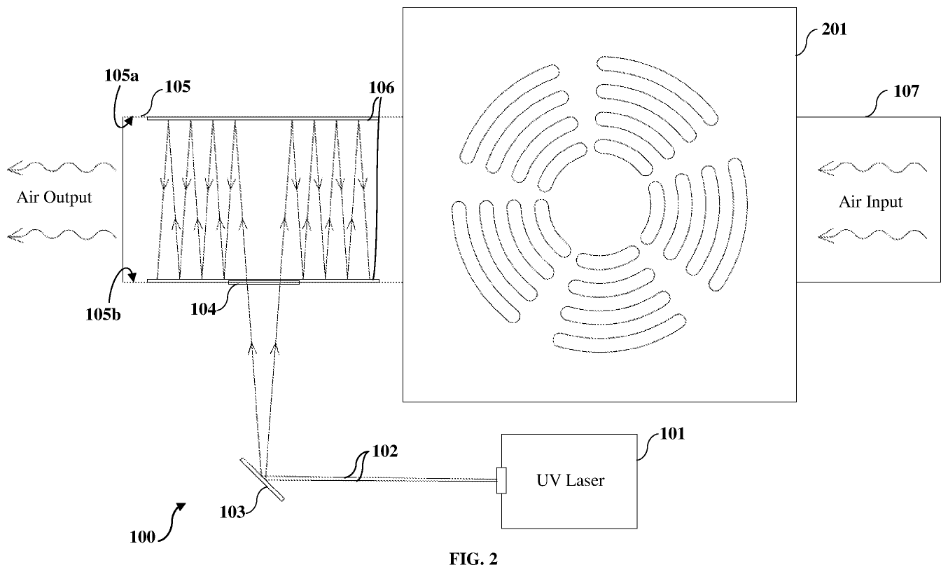

FIG. 2 exemplarily illustrates an embodiment of the disinfection system 100 positioned in communication with an air outlet channel 105 of an airflow system 201 for disinfecting air flowing in the air outlet channel 105. The airflow system 201 is, for example, an air conditioning system, an air exchange system, an air purifier system, an air circulation system, etc., configured or positioned in a local area or an enclosed space such as a room of a residential home, a residential building, an apartment building, an office building, a hospital, an airport, a public location, a commercial and business establishment such as a restaurant, a shopping mall, etc., in a transport vehicle such as a subway train, a bus, etc. The air conveyed from the airflow system 201 may contain pathogens.

The airflow system 201 receives an input of air through an air inlet channel 107 as exemplarily illustrated in FIG. 2. The airflow system 201, for example, an air conditioning system, conditions the input air and conveys the conditioned air into the air outlet channel 105. The disinfection system 100 comprising the ultraviolet laser 101, the light transmission window 104, and the reflectors 103 and 106 is positioned proximal to and in communication with the air outlet channel 105 as exemplarily illustrated in FIG. 2. The high power, ultraviolet laser beam 102 generated by the disinfection system 100, reflected by the first reflector 103, and thereafter reflected multiple times by the second reflectors 106 within and throughout the length of the air outlet channel 105 in multiple directions, irradiates and disinfects the conditioned air flowing through the air outlet channel 105 of the airflow system 201 in a short time as disclosed in the description of FIG. 1A. The air output from the air outlet channel 105 is disinfected by ultraviolet laser light of the high power, ultraviolet laser beam 102 and is free of pathogens.

FIG. 3 exemplarily illustrates an embodiment of the disinfection system 100 comprising an additional reflector 108 positioned in communication with an air outlet channel 105 for disinfecting air flowing in the air outlet channel 105. In an embodiment, the disinfection system 100 further comprises one or more third reflectors 108 positioned in a free space optical connection to the first reflector 103 and the second reflectors 106. The third reflector 108 is operably coupled to one or more of the internal surfaces 105a, 105b, etc., of an airflow channel, for example, the air outlet channel 105, of the airflow system 201 exemplarily illustrated in FIG. 2. In an embodiment, a mount 301 is attached to the internal surface 105a of the air outlet channel 105 as exemplarily illustrated in FIG. 3. The third reflector 108 is attached to the mount 301 and configured to extend from the internal surface 105a of the air outlet channel 105 via the mount 301 as exemplarily illustrated in FIG. 3. The third reflector 108 is of a geometrical shape, for example, a circular shape, a square shape, a rectangular shape, etc., with a length ranging, for example, from about 5 mm to about 10 meters. The thickness of the third reflector 108 is, for example, from about 0.1 mm to about 100 mm. The third reflector 108 is optically aligned with the second reflectors 106 in the air outlet channel 105 as exemplarily illustrated in FIG. 3. The third reflector 108 is configured to reflect and direct the high power, ultraviolet laser beam 102 reflected by the first reflector 103 to the second reflectors 106 to further increase the contact, the utilization, and the interaction of the reflected ultraviolet laser beam 102 with the air flowing in the air outlet channel 105.

Air, that may contain pathogens, flows through the air outlet channel 105 as exemplarily illustrated in FIG. 3. The ultraviolet laser 101 and the first reflector 103 of the disinfection system 100 are positioned outside the air outlet channel 105 as exemplarily illustrated in FIG. 3. The light transmission window 104 of the disinfection system 100 is attached on the internal surface 105b of the air outlet channel 105 as exemplarily illustrated in FIG. 3. The second reflectors 106 are attached to the internal surfaces 105a, 105b, etc., of the air outlet channel 105 respectively as exemplarily illustrated in FIG. 3. The ultraviolet laser 101 generates and directs a high power, ultraviolet laser beam 102 with a wavelength in a range of, for example, about 150 nm to about 300 nm, to the first reflector 103. The first reflector 103 reflects the high power, ultraviolet laser beam 102 into the air outlet channel 105 via the light transmission window 104. The light transmission window 104 passes the high power, ultraviolet laser beam 102 to the third reflector 108 mounted in the air outlet channel 105. The third reflector 108 further reflects the high power, ultraviolet laser beam 102 to the second reflectors 106 on the internal surfaces 105a, 105b, etc., of the air outlet channel 105 respectively. The second reflectors 106 reflect the high power, ultraviolet laser beam 102 onto the air flowing through the air outlet channel 105. The reflectors 103, 106, and 108 redirect the high power, ultraviolet laser beam 102 in multiple directions without changing the wavelength of the high power, ultraviolet laser beam 102. The multiple reflections of the high power, ultraviolet laser beam 102 by the third reflector 108 and thereafter by the second reflectors 106 increase the contact, the utilization, and the interaction of the high power, ultraviolet laser beam 102 with the air flowing through the air outlet channel 105, thereby irradiating and disinfecting the air in a short time.

FIG. 4A exemplarily illustrates an embodiment of the disinfection system 100 positioned in communication with an air inlet channel 107 of an airflow system 201 for disinfecting air flowing in the air inlet channel 107. The disinfection system 100 comprises the ultraviolet laser 101 and the reflectors 103 and 106 as disclosed in the description of FIG. 1A. The disinfection system 100 irradiates and disinfects air flowing through an airflow channel, for example, the air inlet channel 107 exemplarily illustrated in FIG. 4A, of the airflow system 201 from pathogens using the ultraviolet laser 101. The air inlet channel 107 is configured to allow entry of air into the airflow system 201. As exemplarily illustrated in FIG. 4A, the disinfection system 100 comprising the ultraviolet laser 101 and the reflectors 103 and 106 is positioned proximal to and in a free space optical connection to the air inlet channel 107 through which air enters and flows from one end 107c to another end 107d of the air inlet channel 107. The air entering the air inlet channel 107 of the airflow system 201 may contain pathogens.

The ultraviolet laser 101, for example, a pulsed ultraviolet laser, and in an embodiment, a continuous wave ultraviolet laser, as disclosed in the description of FIG. 1A, is positioned proximal to the air inlet channel 107 outside the airflow system 201 as exemplarily illustrated in FIG. 4A. The ultraviolet laser 101 generates and directs a high power, ultraviolet laser beam 102 of wavelength in a range, for example, from about 150 nm to about 300 nm, into the air inlet channel 107 via a first reflector 103 for inactivating and/or killing the pathogens contained in the air flowing through the air inlet channel 107. The first reflector 103, for example, a reflective mirror, is positioned in a free space optical connection to the ultraviolet laser 101. In an embodiment, more than one first reflector 103 is positioned in a free space optical connection to the ultraviolet laser 101 for reflecting and directing the high power, ultraviolet laser beam 102 into the air inlet channel 107 of the airflow system 201. As exemplarily illustrated in FIG. 4A, the first reflector 103 is positioned proximal to the air inlet channel 107 outside the airflow system 201. The ultraviolet laser 101 directs the high power, ultraviolet laser beam 102 to the first reflector 103, which in turn, reflects the high power, ultraviolet laser beam 102 into the air inlet channel 107 of the airflow system 201. One or more second reflectors 106 are attached to internal surfaces 107a, 107b, etc., of the air inlet channel 107. For example, four second reflectors 106a, 106b, 106c, and 106d are positioned on the internal surfaces 107a, 107b, 107e, and 107f of the air inlet channel 107 respectively, as exemplarily illustrated in FIG. 4B. The second reflectors 106 are, for example, reflective mirrors, reflective mirror films, reflective mirror plates, etc., attached to the internal surfaces 107a, 107b, etc., of the air inlet channel 107, for example, using adhesive materials, fasteners, etc. In an embodiment, the second reflectors 106 are configured to be positioned on other internal surfaces and at the ends of the air inlet channel 107 for reflecting the high power, ultraviolet laser beam 102 within the air inlet channel 107 multiple times.

In an embodiment, the disinfection system 100 further comprises a light transmission window 104 positioned at a predetermined location on the air inlet channel 107, similar to the light transmission window 104 positioned at a predetermined location on the air outlet channel 105 as disclosed in the description of FIG. 1A. For example, the light transmission window 104 is centrally positioned, proximal to the internal surface 107b of the air inlet channel 107 as exemplarily illustrated in FIG. 4A. The light transmission window 104 is attached to the internal surface 107b of the air inlet channel 107, for example, using adhesive materials, fasteners, etc. In an example, the light transmission window 104 is made of glass configured to optimally transmit the high power, ultraviolet laser beam 102 reflected by the first reflector 103 into the air inlet channel 107. The second reflectors 106 are positioned in a free space optical connection to the first reflector 103 via the light transmission window 104. The second reflectors 106 are installed in optical alignment with the light transmission window 104 to fully utilize the incident high power, ultraviolet laser beam 102 and maximize interaction of the high power, ultraviolet laser beam 102 with the air flowing through the air inlet channel 107 to disinfect the air. The first reflector 103 is optically aligned with the centrally positioned light transmission window 104 as exemplarily illustrated in FIG. 4A. The light transmission window 104 passes, directs, and transmits the high power, ultraviolet laser beam 102 generated by the ultraviolet laser 101 and reflected by the first reflector 103 to the second reflectors 106 in the air inlet channel 107.

The second reflectors 106 reflect the reflected ultraviolet laser beam 102 multiple times within and throughout the length of the air inlet channel 107 in multiple directions, for example, a backward direction, a forward direction, an upward direction, a downward direction, etc., with the reflected ultraviolet laser beam 102 forming an ultraviolet laser beam tunnel to increase contact, utilization, and interaction of the reflected ultraviolet laser beam 102 with the air flowing in the air inlet channel 107 for irradiating and disinfecting the air in a short time. In an example, the time range for exposing the airflow to the ultraviolet laser beam 102 in the air inlet channel 107 for irradiating and disinfecting the air in the airflow in the air inlet channel 107 is about 1 second to about 100 minutes. Depending on the actual air flow volume and the output power of the ultraviolet laser 101, the time for irradiating and disinfecting the air in the air inlet channel 107 is, for example, about 1 second to about 30 minutes. The high power, ultraviolet laser beam 102 reflected by the first reflector 103 into the air inlet channel 107 via the light transmission window 104 is reflected multiple times between the second reflectors 106. The multi-reflecting high power, ultraviolet laser beam 102 within the air inlet channel 107 exposes the air flowing through the air inlet channel 107 to high power, ultraviolet radiation, for example, ultraviolet-C (UVC) radiation, of wavelength in a range of, for example, about 150 nm to about 300 nm, thereby irradiating the air and disinfecting the air in a short time. The irradiation of the air flowing through the air inlet channel 107 by the multi-reflecting high power, ultraviolet laser beam 102 inactivates and/or kills the pathogens.

As exemplarily illustrated in FIG. 4A, the airflow system 201 receives an input airflow through the air inlet channel 107 as exemplarily illustrated in FIG. 4A. The high power, ultraviolet laser beam 102 generated by the disinfection system 100, reflected by the first reflector 103, and thereafter reflected multiple times by the second reflectors 106 within and throughout the length of the air inlet channel 107 in multiple directions, irradiates and disinfects the air flowing through the air inlet channel 107 of the airflow system 201 in a short time. The air output from the air inlet channel 107 is disinfected by ultraviolet laser light of the high power, ultraviolet laser beam 102. The airflow system 201, for example, an air conditioning system, receives the disinfected air from the air inlet channel 107 and conditions the disinfected air. The airflow system 201 then conveys the conditioned, disinfected air through the air outlet channel 105 for delivery to a local area or an enclosed space.

FIG. 4B exemplarily illustrates a cross-sectional view of the air inlet channel 107 taken along a section B-B shown in FIG. 4A. In an embodiment, the second reflectors 106a, 106b, 106c, and 106d are positioned inside the surfaces 107a, 107b, 107e, and 107f of the air inlet channel 107 respectively. The cross-sectional view in FIG. 4B exemplarily illustrates four second reflectors 106a, 106b, 106c, and 106d positioned on the internal surfaces 107a, 107b, 107e, and 107f of the air inlet channel 107 respectively. In another embodiment, the second reflectors 106a, 106b, 106c, and 106d cover the entire internal surface area of the air inlet channel 107. In an embodiment, the first reflector 103 reflects the incoming high power, ultraviolet laser beam 102 and redirects the divergent, high power, ultraviolet laser beam 102 into the air inlet channel 107 via the light transmission window 104. The second reflectors 106a, 106b, 106c, and 106d reflect the divergent, high power, ultraviolet laser beam 102 multiple times in multiple directions, for example, opposing directions such as upward and downward directions, back and forth directions, etc., from the internal surfaces 107a, 107e to other internal surfaces 107b, 107f of the air inlet channel 107 as exemplarily illustrated in FIG. 4B. In an example, the second reflectors 106a and 106b reflect the high power, ultraviolet laser beam 102 vertically in upward and downward directions from one internal surface 107a to another internal surface 107b of the air inlet channel 107 and vice versa. Similarly, the second reflectors 106c and 106d reflect the high power, ultraviolet laser beam 102 horizontally in back and forth directions from one internal surface 107e to another internal surface 107f of the air inlet channel 107 and vice versa. The second reflectors 106a, 106b, 106c, and 106d, therefore, reflect the high power, ultraviolet laser beam 102 all around the inside of the air inlet channel 107, thereby forming an ultraviolet laser beam tunnel within the air inlet channel 107 for increasing contact and maximizing the interaction between the air flowing through the air inlet channel 107 and the high power, ultraviolet laser beam 102.

As exemplarily illustrated in FIG. 4B, the ultraviolet laser beam 102 at the entrance of the light transmission window 104 is directed to the reflector 106a on the upper internal surface 107a of the air inlet channel 107 in opposing directions, from where the ultraviolet laser beam 102 incident on the reflector 106a is reflected in the general direction of the reflector 106b on the lower internal surface 107b of the air inlet channel 107. This reflection of the ultraviolet laser beam 102 proceeds in opposing directions to the reflectors 106c and 106d on the internal surfaces 107e and 107f of the air inlet channel 107 respectively, from where the ultraviolet laser beam 102 is reflected between the reflectors 106b and 106a multiple times. The ultraviolet laser beam 102 reflected from the reflector 106a located on the upper internal surface 107a of the air inlet channel 107 to the reflector 106b located on the lower internal surface 107b and back to the reflector 106a on the upper internal surface 107a of the air inlet channel 107, permeates the entire space 107g within the air inlet channel 107 and contacts the air flowing through the air inlet channel 107. The ultraviolet laser beam 102 in the air inlet channel 107 inactivates the pathogens in the air flowing through the air inlet channel 107.

The reflection of the divergent, high power, ultraviolet laser beam 102 multiple times in multiple directions from internal surfaces 107a, 107e to the other internal surfaces 107b, 107f of the air inlet channel 107 provides maximum direct exposure of the air flowing through the air inlet channel 107 to the multi-reflecting high power, ultraviolet laser beam 102, thereby allowing the air to receive a substantial dosage of the ultraviolet radiation, for example, the ultraviolet-C (UVC) radiation, to facilitate an irreversible chemical reaction to the deoxyribonucleic acid (DNA) of the pathogens in the airflow through the air inlet channel 107 and a disinfection of the air in the air inlet channel 107.

FIG. 5 exemplarily illustrates an embodiment of the disinfection system 100 comprising an additional reflector 108 positioned in communication with an air inlet channel 107 for disinfecting air flowing in the air inlet channel 107. In an embodiment, the disinfection system 100 further comprises one or more third reflectors 108 positioned in a free space optical connection to the first reflector 103 and the second reflectors 106 as disclosed in the description of FIG. 3. The third reflector 108 is operably coupled to one or more of the internal surfaces 107a, 107b, etc., of an airflow channel, for example, the air inlet channel 107, of the airflow system 201. In an embodiment, a mount 501 is attached to the internal surface 107a of the air inlet channel 107 as exemplarily illustrated in FIG. 5. The third reflector 108 is attached to the mount 501 and configured to extend from the internal surface 107a of the air inlet channel 107 via the mount 501 as exemplarily illustrated in FIG. 5. The third reflector 108 is optically aligned with the second reflectors 106 in the air inlet channel 107 as exemplarily illustrated in FIG. 5. The third reflector 108 is configured to reflect and direct the high power, ultraviolet laser beam 102 reflected by the first reflector 103 to the second reflectors 106 to further increase contact, utilization, and interaction of the reflected ultraviolet laser beam 102 with the air flowing in the air inlet channel 107.

Air, that may contain pathogens, flows through the air inlet channel 107 as exemplarily illustrated in FIG. 5. The ultraviolet laser 101 and the first reflector 103 of the disinfection system 100 are positioned outside the air inlet channel 107 as exemplarily illustrated in FIG. 5. The light transmission window 104 of the disinfection system 100 is attached on the internal surface 107b of the air inlet channel 107 as exemplarily illustrated in FIG. 5. The second reflectors 106 are attached to the internal surfaces 107a, 107b, etc., of the air inlet channel 107 as exemplarily illustrated in FIG. 5. The ultraviolet laser 101 generates and directs a high power, ultraviolet laser beam 102 with a wavelength in a range of, for example, about 150 nm to about 300 nm, to the first reflector 103. The first reflector 103 reflects the high power, ultraviolet laser beam 102 into the air inlet channel 107 via the light transmission window 104. The light transmission window 104 passes the high power, ultraviolet laser beam 102 to the third reflector 108 mounted in the air inlet channel 107. The third reflector 108 further reflects the high power, ultraviolet laser beam 102 to the second reflectors 106 on the internal surfaces 107a, 107b, etc., of the air inlet channel 107. The second reflectors 106 reflect the high power, ultraviolet laser beam 102 onto the air flowing through the air inlet channel 107. The multiple reflections of the high power, ultraviolet laser beam 102 by the third reflector 108 and thereafter by the second reflectors 106 in multiple directions forms an ultraviolet laser beam tunnel in the air inlet channel 107, which increases the contact, the utilization, and the interaction of the high power, ultraviolet laser beam 102 with the air flowing through the air inlet channel 107, thereby irradiating and disinfecting the air in a short time. The disinfected air from the air inlet channel 107 then enters the airflow system 201, which conditions and circulates the disinfected air into a local area or an enclosed space.

FIG. 6 exemplarily illustrates an embodiment of the disinfection system 100 positioned in communication with an air inlet channel 107 and an air outlet channel 105 of an airflow system 201 for disinfecting air flowing in the air inlet channel 107 and the air outlet channel 105. In this embodiment, the disinfection system 100 comprises two ultraviolet lasers 101 and two first reflectors 103, where a first ultraviolet laser 101 and one first reflector 103 are positioned outside the air inlet channel 107 and a second ultraviolet laser 101 and another first reflector 103 are positioned outside the air outlet channel 105 as exemplarily illustrated in FIG. 6. One light transmission window 104 is attached on the internal surface 107b of the air inlet channel 107 and another light transmission window 104 is attached on the internal surface 105b of the air outlet channel 105 as exemplarily illustrated in FIG. 6. One set of second reflectors 106 are attached to the internal surfaces 107a, 107b, etc., of the air inlet channel 107 as disclosed in the description of FIG. 4B, and another set of second reflectors 106 are attached to the internal surfaces 105a, 105b, etc., of the air outlet channel 105 as disclosed in the description of FIG. 1B. The airflow system 201 receives air through the air inlet channel 107 and exhausts air through the air outlet channel 105.

The first ultraviolet laser 101 positioned outside the air inlet channel 107 generates and directs a high power, ultraviolet laser beam 102 with a wavelength in a range of, for example, about 150 nm to about 300 nm, to the first reflector 103. The first reflector 103 reflects the high power, ultraviolet laser beam 102 into the air inlet channel 107 via the light transmission window 104. The light transmission window 104 passes the high power, ultraviolet laser beam 102 to the second reflectors 106 on the internal surfaces 107a, 107b, etc., of the air inlet channel 107. The second reflectors 106 reflect the high power, ultraviolet laser beam 102 onto the air flowing through the air inlet channel 107. The multiple reflections of the high power, ultraviolet laser beam 102 by the second reflectors 106 in multiple directions forms an ultraviolet laser beam tunnel in the air inlet channel 107 to increase contact, utilization, and interaction of the high power, ultraviolet laser beam 102 with the air flowing through the air inlet channel 107, thereby irradiating and disinfecting the air in a short time. The disinfected air from the air inlet channel 107 then enters the airflow system 201.

The airflow system 201, for example, an air conditioning system, conditions the input air and conveys the conditioned air into the air outlet channel 105. The second ultraviolet laser 101 positioned outside the air outlet channel 105 generates and directs a high power, ultraviolet laser beam 102 with a wavelength in a range of, for example, about 150 nm to about 300 nm, to the first reflector 103. The first reflector 103 reflects the high power, ultraviolet laser beam 102 into the air outlet channel 105 via the light transmission window 104. The light transmission window 104 passes the high power, ultraviolet laser beam 102 to the second reflectors 106 on the internal surfaces 105a, 105b, etc., of the air outlet channel 105. The second reflectors 106 reflect the high power, ultraviolet laser beam 102 onto the air flowing through the air outlet channel 105. The multiple reflections of the high power, ultraviolet laser beam 102 by the second reflectors 106 in multiple directions forms an ultraviolet laser beam tunnel in the air outlet channel 105 to increase contact, utilization, and interaction of the high power, ultraviolet laser beam 102 with the air flowing through the air outlet channel 105, thereby irradiating and disinfecting the air flowing in the air outlet channel 105 in a short time.

The second ultraviolet laser 101 and the reflectors 103 and 106 at the air outlet channel 105 disinfect any residual pathogens that may be present in the air flowing through the air outlet channel 105. The air output from the air outlet channel 105 is, therefore, doubly disinfected by the ultraviolet laser light of the high power, ultraviolet laser beams 102 produced by the two ultraviolet lasers 101 and is free of pathogens. This embodiment of the disinfection system 100 provides maximum direct exposure of the air flowing through the air inlet channel 107 and the air outlet channel 105 to the multi-reflecting high power, ultraviolet laser beams 102 generated by the two ultraviolet lasers 101, thereby allowing the air to receive a substantial dosage of the ultraviolet radiation, for example, the ultraviolet-C (UVC) radiation, to facilitate an irreversible chemical reaction to the deoxyribonucleic acid (DNA) of pathogens in the airflow through the air inlet channel 107 and the air outlet channel 105 and disinfection of the airflow.

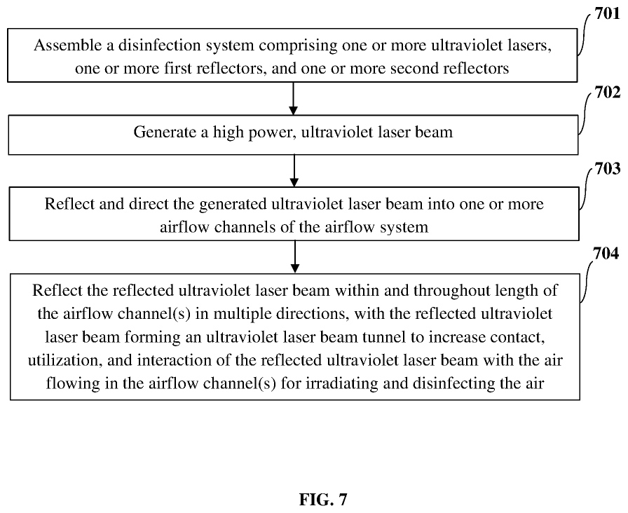

FIG. 7 illustrates a flowchart of an embodiment of a method for disinfecting air in an airflow system 201. In the method disclosed herein, the disinfection system 100 comprising one or more ultraviolet lasers 101 and the reflectors 103 and 106 are assembled 701 as exemplarily illustrated in FIGS. 1A-6. The first reflector 103 is positioned in a free space optical connection to the ultraviolet laser(s) 101, and the second reflectors 106 are positioned in a free space optical connection to the first reflector 103. The ultraviolet laser(s) 101 and the reflectors 103 and 106 are positioned proximal to and in a free space optical connection to the airflow system 201 as disclosed in the descriptions of FIGS. 1A-6. For example, the ultraviolet laser 101 and the first reflector 103 are externally positioned proximal to one or more airflow channels such as the air outlet channel 105 and/or the air inlet channel 107 of the airflow system 201 as exemplarily illustrated in FIGS. 1A-6, and the second reflectors 106 are positioned inside the airflow channel(s) 105 and/or 107. The ultraviolet laser 101 generates 702 a high power, ultraviolet laser beam 102 for inactivating and/or killing pathogens contained in the air flowing through the airflow system 201. The first reflector 103 reflects and directs 703 the generated ultraviolet laser beam 102 into one or more airflow channels, for example, the air outlet channel 105 and/or the air inlet channel 107 of the airflow system 201 as exemplarily illustrated in FIGS. 1A-6. The second reflectors 106, in a free space optical connection to the first reflector 103, reflect 704 the reflected ultraviolet laser beam 102 multiple times within and throughout the length of the airflow channel(s) 105 and/or 107 in multiple directions, with the reflected ultraviolet laser beam 102 forming an ultraviolet laser beam tunnel to increase contact, utilization, and interaction of the reflected ultraviolet laser beam 102 with the air flowing in the airflow channel(s) 105 and/or 107 of the airflow system 201 for irradiating and disinfecting the air. The second reflectors 106 that reflect the high power, ultraviolet laser beam 102 within the airflow channel(s) 105 and/or 107 multiple times in multiple directions maximize the utilization of the ultraviolet laser beam energy by the air flowing through the airflow channel(s) 105 and/or 107 to inactivate and/or kill pathogens in the airflow.

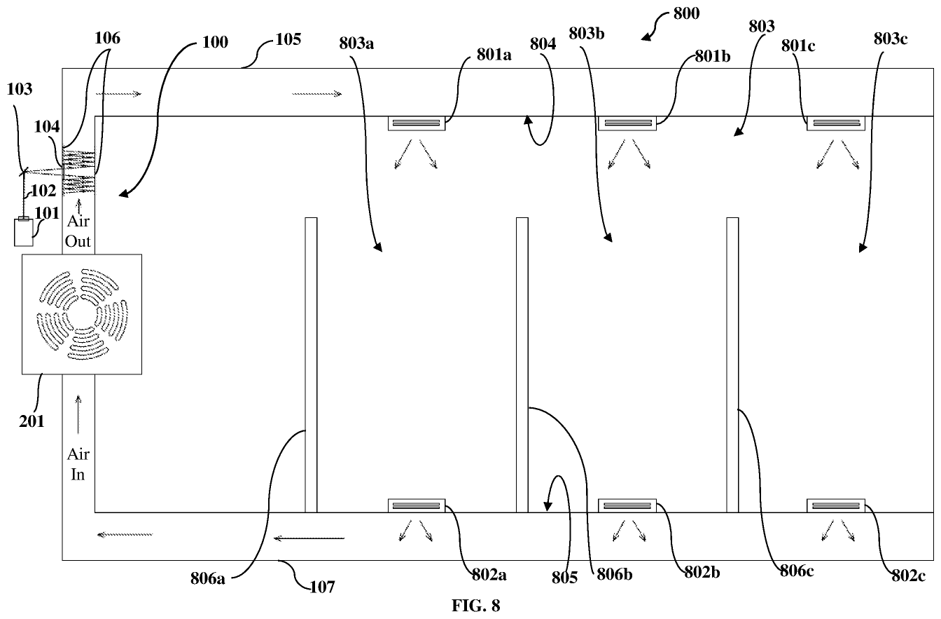

FIG. 8 exemplarily illustrates an embodiment of a system 800 for disinfecting air and for minimizing and/or precluding cross-contamination of the air in an enclosed space 803. In this embodiment, the system 800 comprises an airflow system 201, multiple air supply components 801a, 801b, and 801c, multiple air exhaust components 802a, 802b, and 802c, and the disinfection system 100 disclosed in the descriptions of FIGS. 1A-6. The airflow system 201 is configured to allow a flow of air through airflow channels, for example, an air outlet channel 105 and an air inlet channel 107, into an enclosed space 803. Consider an example where an airflow system 201 such as an air conditioning system comprising an air outlet channel 105 and an air inlet channel 107 is configured to condition an enclosed space 803 such as a room of a residential home, an apartment building, an office building, a hospital, an airport, a commercial and business establishment, etc. The air inlet channel 107 extends below a floor 805 of the enclosed space 803 as exemplarily illustrated in FIG. 8. The air outlet channel 105 extends above a ceiling 804 of the enclosed space 803 as exemplarily illustrated in FIG. 8. The air supply components 801a, 801b, and 801c are operably coupled to and in fluid communication with the air outlet channel 105 of the airflow system 201. In an embodiment, the air supply components 801a, 801b, and 801c are configured as air blowers or fans configured to blow the irradiated and disinfected air output from the disinfection system 100 into enclosed spaces 803a, 803b, and 803c respectively constituting the enclosed space 803. In another embodiment, the air supply components 801a, 801b, and 801c are configured as ports or openings configured to direct the irradiated and disinfected air output from the disinfection system 100 into the enclosed spaces 803a, 803b, and 803c respectively. In an embodiment, the air supply components 801a, 801b, and 801c extend into the enclosed space 803 from the ceiling 804 of the enclosed space 803.

The air exhaust components 802a, 802b, and 802c are operably coupled to and in fluid communication with the air inlet channel 107. In an embodiment, the air exhaust components 802a, 802b, and 802c are configured as air suction devices configured to suction air exhaled by one or more occupants of the enclosed spaces 803a, 803b, and 803c respectively, and exhaust the exhaled air into the air inlet channel 107 of the airflow system 201. In another embodiment, the air exhaust components 802a, 802b, and 802c are configured as ports or openings for exhausting the exhaled air into the air inlet channel 107 of the airflow system 201. In an embodiment, the air exhaust components 802a, 802b, and 802c are positioned inside the enclosed space 803 on the floor 805 of the enclosed space 803. The air exhaust components 802a, 802b, and 802c are configured to receive air exhaled by one or more occupants in the enclosed spaces 803a, 803b, and 803c respectively, and exhaust the exhaled air into the air inlet channel 107 of the airflow system 201 for subsequent irradiation and disinfection by the high power, ultraviolet laser beam 102 generated by the ultraviolet laser 101 positioned, in this example, proximal to the air outlet channel 105. The exhaled air flows through the air inlet channel 107 and enters the airflow system 201. The airflow system 201 conditions the exhaled air and delivers the conditioned air to the air outlet channel 105.

The disinfection system 100 is in operable communication with one or more of the airflow channels, for example, the air inlet channel 107 and/or the air outlet channel 105, of the airflow system 201. In an embodiment as exemplarily illustrated in FIG. 8, the disinfection system 100 is positioned at the air outlet channel 105 of the airflow system 201 as disclosed in the description of FIG. 1A and FIGS. 2-3. In this example, the disinfection system 100 is in operable communication with the air outlet channel 105 of the airflow system 201. For purposes of illustration, the description of FIG. 8 refers to the disinfection system 100 being positioned at the air outlet channel 105 of the airflow system 201 for disinfecting the air flowing out through the air outlet channel 105; however, the scope of the system 800 disclosed herein is not limited to the disinfection system 100 being positioned at the air outlet channel 105 of the airflow system 201, but may be extended to be positioned alternatively at the air inlet channel 107 of the airflow system 201 for disinfecting the air flowing through the air inlet channel 107 as disclosed in the description of FIG. 4A. In another embodiment, the disinfection system 100 is configured to be positioned at both the air outlet channel 105 and the air inlet channel 107 of the airflow system 201 as exemplarily illustrated in FIG. 6, for disinfecting the air flowing through both the air inlet channel 107 and the air outlet channel 105 as disclosed in the description of FIG. 6.

The disinfection system 100 comprises one or more ultraviolet lasers 101, the light transmission window 104, and the reflectors 103 and 106 as disclosed in the descriptions of FIGS. 1A-2. In an embodiment, the disinfection system 100 further comprises a third reflector 108 (not shown in FIG. 8) as disclosed in the descriptions of FIG. 3 and FIG. 5. In this example, the disinfection system 100 irradiates and disinfects the air, that may contain airborne pathogens, flowing through the air outlet channel 105 of the airflow system 201 as disclosed in the descriptions of FIGS. 1A-2. The air supply components 801a, 801b, and 801c are configured to receive and blow the irradiated and disinfected air output from the disinfection system 100, from the air outlet channel 105 of the airflow system 201, into the enclosed spaces 803a, 803b, and 803c respectively, for inhalation by the occupants in the enclosed spaces 803a, 803b, and 803c, while the air exhaust components 802a, 802b, and 802c receive and exhaust the air exhaled by the occupants in the enclosed spaces 803a, 803b, and 803c respectively, into the air inlet channel 107 of the airflow system 201 for subsequent irradiation and disinfection by the high power, ultraviolet laser beam 102 generated by the ultraviolet laser 101 positioned, in this example, proximal to the air outlet channel 105 of the airflow system 201, thereby minimizing and/or precluding cross-contamination of the air in the enclosed space 803.

In an embodiment, the system 800 for disinfecting air and for minimizing and/or precluding cross-contamination of the air in an enclosed space 803 further comprises guide members 806a, 806b, and 806c extending upwardly into the enclosed space 803 from the floor 805 of the enclosed space 803. The guide members 806a, 806b, and 806c are positioned between each of the air supply components 801a, 801b, and 801c and a corresponding one of the air exhaust components 802a, 802b, and 802c. The guide members 806a, 806b, and 806c are configured to allow only a vertical flow of air within the enclosed spaces 803a, 803b, and 803c from the ceiling 804 to the floor 805 of the enclosed spaces 803a, 803b, and 803c and preclude a horizontal flow of the air within the enclosed space 803. The guide members 806a, 806b, and 806c are configured, for example, as separators, that separate the enclosed space 803, for example, into separate enclosed spaces 803a, 803b, and 803c as exemplarily illustrated in FIG. 8, to allow only a vertical flow of air from the ceiling 804 to the floor 805 in each of the enclosed spaces 803a, 803b, and 803c and to preclude a horizontal flow of the air within the enclosed space 803. The air flows from the air supply components 801a, 801b, and 801c into respective enclosed spaces 803a, 803b, and 803c, and the air flows out of the enclosed spaces 803a, 803b, and 803c through the respective air exhaust components 802a, 802b, and 802c.

In an embodiment, the guide members 806a, 806b, and 806c are made of a transparent material configured to provide visibility within the enclosed space 803. The guide members 806a and 806b are configured to guide the irradiated and disinfected air blown by the air supply component 801a to the occupants in the enclosed space 803a for inhalation by the occupants in the enclosed space 803a and to guide the air exhaled by the occupants in the enclosed space 803a into the air exhaust components 802a, thereby minimizing and/or precluding cross-contamination of the air between the enclosed spaces 803a and 803b. Similarly, the guide members 806b and 806c are configured to guide the irradiated and disinfected air blown by the air supply components 801b and 801c to the occupants in the enclosed spaces 803b and 803c respectively, for inhalation by the occupants in the enclosed spaces 803b and 803c, and to guide the air exhaled by the occupants of the enclosed spaces 803b and 803c into the air exhaust components 802b and 802c respectively, thereby minimizing and/or precluding cross-contamination of the air between the enclosed spaces 803a, 803b, and 803c.

Through this system 800, the air exhaled by the occupants of the enclosed space 803a does not flow into the enclosed space 803b for inhalation by the occupants of the space 803b, and the air exhaled by the occupants of the space 803b does not flow into the enclosed spaces 803a and 803c for inhalation by the occupants of the enclosed spaces 803a and 803c, thereby precluding cross-contamination of the air between the occupants in different enclosed spaces 803a, 803b, and 803c. The system 800 exemplarily illustrated in FIG. 8, is configured to ensure that the air exhaled by the occupants in the enclosed space 803 is guided and transferred to the air inlet channel 107 of the airflow system 201 via the air exhaust components 802a, 802b, and 802c so that the exhaled air is not inhaled by other occupants in the enclosed space 803, thereby minimizing and/or precluding cross-contamination of the air in the enclosed space 803 and cross-infection between occupants in the enclosed space 803. The system 800 disclosed herein provides disinfected airflow inside the enclosed space 803, while minimizing and/or preventing cross-contamination of the air by airborne pathogens.

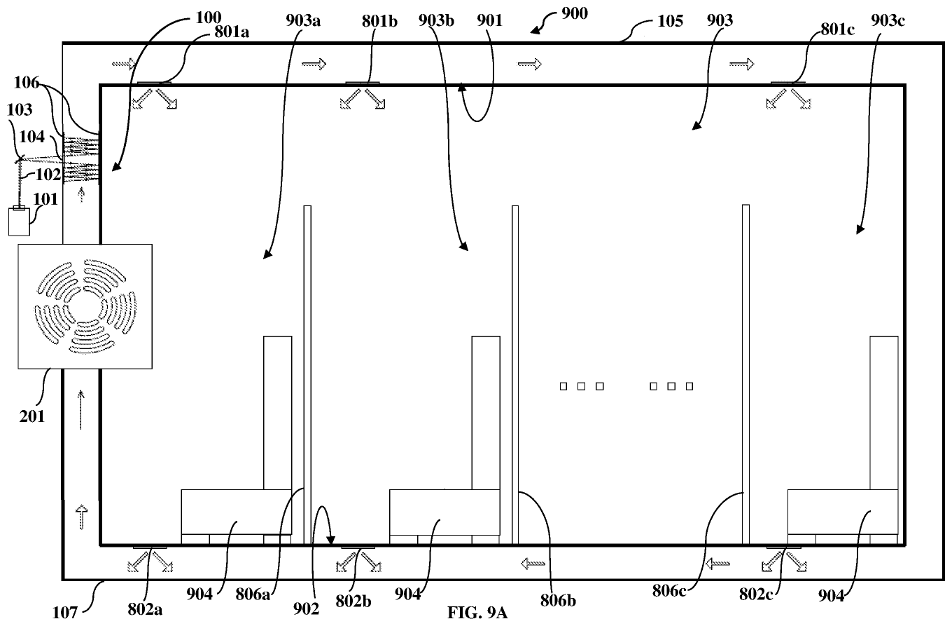

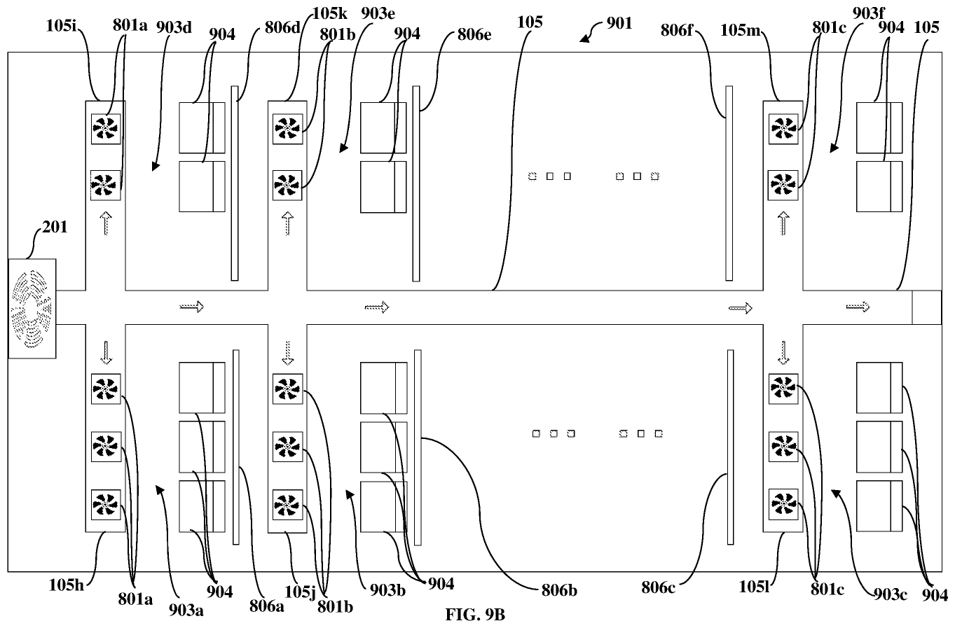

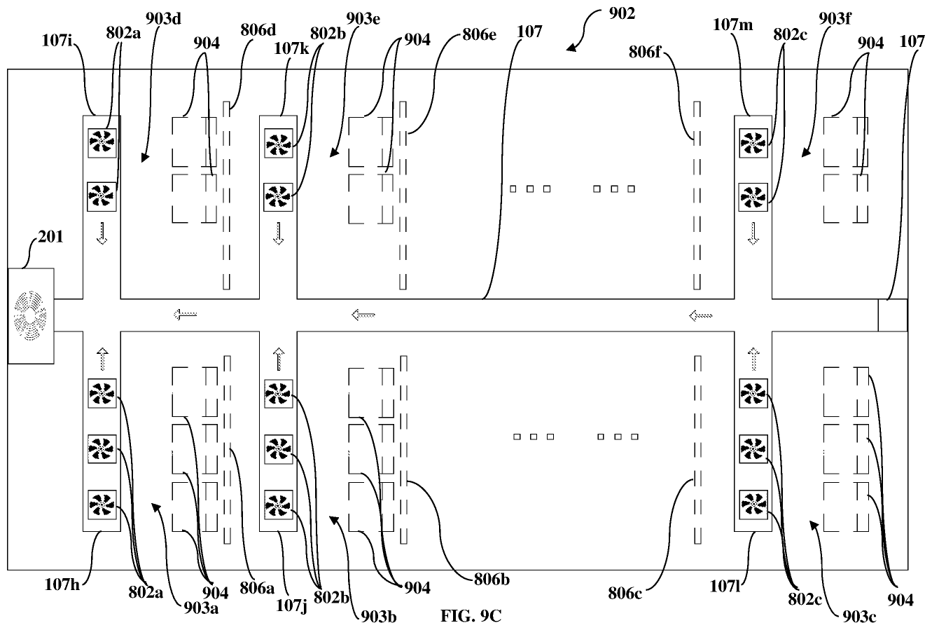

FIG. 9A exemplarily illustrates a side view of an embodiment of a system 900 for disinfecting air and for minimizing and/or precluding cross-contamination of the air in an enclosed space 903, for example, in a transport vehicle. In this embodiment, the system 900 comprises an airflow system 201, multiple air supply components 801a, 801b, and 801c, multiple air exhaust components 802a, 802b, and 802c, guide members 806a, 806b, 806c, etc., and the disinfection system 100 disclosed in the descriptions of FIGS. 1A-6. The airflow system 201 communicates a flow of air through airflow channels, for example, an air outlet channel 105 and an air inlet channel 107, into the enclosed space 903. Consider an example where an airflow system 201 such as an air conditioning system comprising an air outlet channel 105 and an air inlet channel 107 is configured to condition an enclosed space 903 in a transport vehicle such as a bus or a subway train. The air inlet channel 107 extends below a floor 902 of the enclosed space 903 as exemplarily illustrated in FIG. 9A. The air outlet channel 105 extends above a ceiling 901 of the enclosed space 903 as exemplarily illustrated in FIG. 9A. The air supply components 801a, 801b, and 801c are operably coupled to and in fluid communication with the air outlet channel 105 of the airflow system 201. In an embodiment, the air supply components 801a, 801b, and 801c extend into the enclosed space 903 from the ceiling 901 of the enclosed space 903. The air exhaust components 802a, 802b, and 802c are operably coupled to and in fluid communication with the air inlet channel 107. In an embodiment, the air exhaust components 802a, 802b, and 802c are positioned inside the enclosed space 903 on the floor 902 of the enclosed space 903. The air exhaust components 802a, 802b, and 802c are configured to receive air exhaled by one or more occupants of enclosed spaces 903a, 903b, and 903c respectively, for example, passengers sitting on seats 904 of the bus, and exhaust the exhaled air into the air inlet channel 107 of the airflow system 201 for subsequent irradiation and disinfection by the high power, ultraviolet laser beam 102 generated by the ultraviolet laser 101 positioned, in this example, proximal to the air outlet channel 105. The exhaled air flows through the air inlet channel 107 and enters the airflow system 201. The airflow system 201 conditions the exhaled air and conveys the conditioned air into the air outlet channel 105.

The disinfection system 100 is in operable communication with one or more of the airflow channels, for example, the air inlet channel 107 and/or the air outlet channel 105, of the airflow system 201. In an embodiment as exemplarily illustrated in FIG. 9A, the disinfection system 100 is positioned at the air outlet channel 105 of the airflow system 201 as disclosed in the descriptions of FIG. 1A and FIGS. 2-3. In this example, the disinfection system 100 is in operable communication with the air outlet channel 105 of the airflow system 201. The disinfection system 100 comprises one or more ultraviolet lasers 101, the light transmission window 104, and the reflectors 103 and 106 as disclosed in the descriptions of FIGS. 1A-2. In this example, the disinfection system 100 irradiates and disinfects the air flowing through the air outlet channel 105 of the airflow system 201 as disclosed in the descriptions of FIGS. 1A-2. The air supply components 801a, 801b, and 801c blow the irradiated and disinfected air output from the disinfection system 100, from the air outlet channel 105 of the airflow system 201, into the enclosed spaces 903a, 903b, and 903c respectively, for inhalation by the occupants of the enclosed spaces 903a, 903b, and 903c, while the air exhaust components 802a, 802b, and 802c receive and exhaust the air exhaled by the occupants of the enclosed spaces 903a, 903b, and 903c respectively, into the air inlet channel 107 of the airflow system 201, thereby minimizing and/or precluding cross-contamination of the air in the enclosed space 903.

As exemplarily illustrated in FIG. 9A, the guide members 806a, 806b, 806c, etc., extend upwardly into the enclosed space 903 from the floor 902 of the enclosed space 903. The guide members 806a, 806b, 806c, etc., are positioned between each of the air supply components 801a, 801b, and 801c and a corresponding one of the air exhaust components 802a, 802b, and 802c. The guide members 806a, 806b, 806c, etc., are configured to allow only a vertical flow of the air within the enclosed spaces 903a, 903b, and 903c from the ceiling 901 to the floor 902 of the enclosed spaces 903a, 903b, and 903c and preclude a horizontal flow of the air within the enclosed space 903. The guide members 806a, 806b, 806c, etc., are configured, for example, as separators, that separate the enclosed space 903, for example, into separate enclosed spaces 903a, 903b, 903c, etc., as exemplarily illustrated in FIGS. 9A-9C, to allow only a vertical flow of air from the ceiling 901 to the floor 902 in each of the enclosed spaces 903a, 903b, 903c, etc., and to preclude a horizontal flow of the air within the enclosed space 903. In an embodiment, the guide members 806a and 806b are positioned behind seats 904 in the transport vehicle, for example, the bus. The seats 904 are positioned in rows, for example, in a typical bus layout. In an embodiment, the guide members 806a, 806b, 806c, etc., are made of a transparent material configured to provide visibility between the enclosed spaces 903a, 903b, 903c, etc.

FIG. 9B exemplarily illustrates a top view of the ceiling 901 of the enclosed space 903 shown in FIG. 9A. In an embodiment, the guide members 806a, 806b, 806c, 806d, 806e, and 806f are configured, for example, as separators, that separate the enclosed space 903 into separate enclosed spaces 903a, 903b, 903c, 903d, 903e, and 903f respectively. In an example, the enclosed space 903b is delimited by the guide members 806a and 806b, and the enclosed space 903e is delimited by the guide members 806d and 806e as exemplarily illustrated in FIG. 9B. Moreover, FIG. 9B exemplarily illustrates the guide members 806a, 806b, 806d, 806e, etc., positioned behind the seats 904 in the transport vehicle. Furthermore, FIG. 9B exemplarily illustrates airflow paths indicated by block arrows from the airflow system 201, that is, the air conditioning system of the transport vehicle, to the air outlet channel 105 and branches 105h, 105i, 105j, 105k, 105l, and 105m of the air outlet channel 105. The air outlet channel 105 and its branches 105h, 105i, 105j, 105k, 105l, and 105m extend above the ceiling 901 of the enclosed space 903. The branches 105h, 105l, 105j, 105k, 105l, and 105m of the air outlet channel 105 extend above the respective enclosed spaces 903a, 903d, 903b, 903e, 903c, and 903f. Sets of air supply components 801a, 801b, and 801c are arranged along the branches 105h, 105i, and 105j, 105k, and 105l, 105m of the air outlet channel 105 for the respective enclosed spaces 903a, 903d, 903b, 903e, 903c, and 903f.

In an example, three air supply components 801a are operably coupled to the branch 105h extending above the ceiling 901 of the enclosed space 903a, and a pair of air supply components 801a are operably coupled to the branch 105l extending above the ceiling 901 of the enclosed space 903d as illustrated in FIG. 9B. In another example, three air supply components 801b are operably coupled to the branch 105j extending above the ceiling 901 of the enclosed space 903b, and a pair of air supply components 801b are operably coupled to the branch 105k extending above the ceiling 901 of the enclosed space 903e as illustrated in FIG. 9B. In another example, three air supply components 801c are operably coupled to the branch 105l extending above the ceiling 901 of the enclosed space 903c, and a pair of air supply components 801c are operably coupled to the branch 105m extending above the ceiling 901 of the enclosed space 903f as illustrated in FIG. 9B. In an embodiment, the air supply components 801a, 801b, and 801c are configured as air blowers or fans configured to blow the irradiated and disinfected air output from the disinfection system 100 into the enclosed spaces 903a, 903d, 903b, 903e, 903c, and 903f. In another embodiment, the air supply components 801a, 801b, and 801c are configured as ports or openings configured to direct the irradiated and disinfected air output from the disinfection system 100 into the enclosed spaces 903a, 903d, 903b, 903e, 903c, and 903f.

FIG. 9C exemplarily illustrates a cutaway view, showing a top view of the floor 902 of the enclosed space 903 shown in FIG. 9A. FIG. 9C exemplarily illustrates airflow paths indicated by block arrows from the air inlet channel 107 and branches 107h, 107i, 107j, 107k, 1071, and 107m of the air inlet channel 107 to the airflow system 201, for example, the air conditioning system, of the transport vehicle such as a bus. The air inlet channel 107 and its branches 107h, 107i, 107j, 107k, 1071, and 107m extend below the floor 902 of the enclosed space 903. The branches 107h, 107i, 107j, 107k, 1071, and 107m of the air inlet channel 107 extend below the respective enclosed spaces 903a, 903d, 903b, 903e, 903c, and 903f. Sets of air exhaust components 802a, 802b, and 802c are arranged along the branches 107h, 107i, and 107j, 107k, and 107l, 107m of the air inlet channel 107 for the respective enclosed spaces 903a, 903d, 903b, 903e, 903c, and 903f. For example, three air exhaust components 802a are operably coupled to the branch 107h extending below the floor 902 of the enclosed space 903a, and a pair of air exhaust components 802a are operably coupled to the branch 107i extending below the floor 902 of the enclosed space 903d as illustrated in FIG. 9C. In another example, three air exhaust components 802b are operably coupled to the branch 107j extending below the floor 902 of the enclosed space 903b, and a pair of air exhaust components 802b are operably coupled to the branch 107k extending below the floor 902 of the enclosed space 903e as illustrated in FIG. 9C. In another example, three air exhaust components 802c are operably coupled to the branch 107l extending below the floor 902 of the enclosed space 903c, and a pair of air exhaust components 802c are operably coupled to the branch 107m extending below the floor 902 of the enclosed space 903f as illustrated in FIG. 9C. In an embodiment, the air exhaust components 802a, 802b, and 802c are configured as air suction devices or fans configured to suction waste air or exhaled air from the enclosed spaces 903a, 903d, 903b, 903e, 903c, and 903f into the air inlet channel 107 via the respective branches 107h, 107i, 107j, 107k, 1071, and 107m. In another embodiment, the air exhaust components 802a, 802b, and 802c are configured as ports or openings for exhausting the exhaled air into the air inlet channel 107.

As exemplarily illustrated in FIGS. 9A-9B, the air supply components 801a, 801b, and 801c blow the irradiated and disinfected air output from the disinfection system 100 into respective enclosed spaces 903a, 903d, and 903b, 903e, and 903c, 903f. The air exhaled by occupants, for example, passengers sitting on the seats 904 of the bus, flows out of the enclosed spaces 903a, 903d, and 903b, 903e, and 903c, 903f through the respective air exhaust components 802a, 802b, and 802c. In an example, the guide members 806a and 806b guide the irradiated and disinfected air blown by the air supply components 801b to the occupants in the enclosed space 903b exemplarily illustrated in FIG. 9B, for inhalation by the occupants in the enclosed space 903b, and guide the air exhaled by the occupants in the enclosed space 903b into the air exhaust components 802b exemplarily illustrated in FIG. 9C, thereby minimizing and/or precluding cross-contamination of the air, for example, between the enclosed spaces 903b and 903a and between the enclosed spaces 903b and 903e. Similarly, the guide members 806d and 806e guide the irradiated and disinfected air blown by the air supply components 801b to the occupants in the enclosed space 903e exemplarily illustrated in FIG. 9B, for inhalation by the occupants in the enclosed space 903e, and guide the air exhaled by the occupants in the enclosed space 903e into the air exhaust components 802b exemplarily illustrated in FIG. 9C, thereby minimizing and/or precluding cross-contamination of the air between the enclosed spaces 903e and 903d and between the enclosed spaces 903e and 903b.

Through the system 900, in an example, the air exhaled by the occupants of the enclosed space 903a does not flow into the enclosed space 903b for inhalation by the occupants of the enclosed space 903b, and the air exhaled by the occupants of the space 903b does not flow into the enclosed spaces 903a and 903c for inhalation by the occupants of the spaces 903a and 903c, thereby precluding cross-contamination of the air between the occupants in different enclosed spaces 903a, 903b, and 903c. The system 900 exemplarily illustrated in FIGS. 9A-9C, ensures that the air exhaled by the occupants in the enclosed spaces 903a, 903d, and 903b, 903e, and 903c, 903f that constitute the enclosed space 903 is guided and transferred to the air inlet channel 107 of the airflow system 201 via the respective air exhaust components 802a, 802b, and 802c so that the exhaled air is not inhaled by other occupants in the surrounding enclosed spaces, thereby minimizing and/or precluding cross-contamination of the air in the enclosed space 903 and cross-infection between occupants in the enclosed space 903. The system 900 produces top-to-down ultraviolet-C (UVC) laser-disinfected airflow with a large volume, airflow system 201, for example, from the air supply components 801a, 801b, and 801c at the ceiling 901 of the enclosed space 903 towards the air exhaust components 802a, 802b, and 802c on the floor 902 of the enclosed space 903 for minimizing and/or precluding cross-contamination of the air in the enclosed space 903. Through the system 900, each passenger in a seat 904 only inhales fresh air received from the air supply components 801a, 801b, and 801c at the ceiling 901 of the enclosed space 903. The air exhaled by each passenger is directed to the air exhaust components 802a, 802b, and 802c on the floor 902 of the enclosed space 903, near the seats 904.

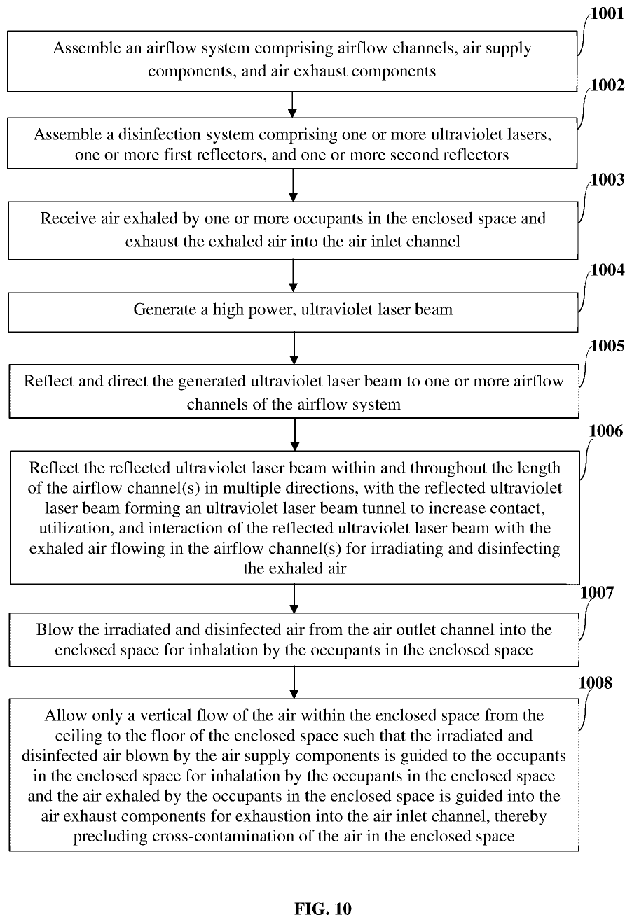

FIG. 10 illustrates a flowchart of an embodiment of a method for disinfecting air and for minimizing and/or precluding cross-contamination of the air in an enclosed space, for example, 803 shown in FIG. 8. In the method disclosed herein, an airflow system 201 comprising airflow channels, for example, an air inlet channel 107 and an air outlet channel 105; multiple air supply components 801a, 801b, and 801c, multiple air exhaust components 802a, 802b, and 802c, and guide members 806a, 806b, 806c, etc., as exemplarily illustrated in FIG. 8 and as disclosed in the description of FIG. 8, is assembled 1001. Furthermore, in the method disclosed herein, the disinfection system 100 comprising the ultraviolet laser 101, the light transmission window 104, and the reflectors 103 and 106 as exemplarily illustrated in FIG. 8 and as disclosed in the descriptions of FIG. 8, is also assembled 1002. Consider an example where multiple occupants gather in enclosed spaces 803a, 803b, and 803c that constitute an enclosed space 803 exemplarily illustrated in FIG. 8. The air exhaust components 802a, 802b, and 802c of the airflow system 201 receive 1003 air exhaled by the occupants in the enclosed spaces 803a, 803b, and 803c respectively, and exhaust the exhaled air into the air inlet channel 107 of the airflow system 201. The exhaled air, that may contain pathogens, flows through the air inlet channel 107 and enters the airflow system 201. The airflow system 201 conditions and conveys the exhaled air into the air outlet channel 105.

The ultraviolet laser 101 of the disinfection system 100 positioned proximal to and in a free space optical connection to one or more of the airflow channels, for example, the air outlet channel 105, generates 1004 a high power, ultraviolet laser beam 102 for inactivating and/or killing airborne pathogens contained in the exhaled air flowing through the air outlet channel 105. The first reflector 103 of the disinfection system 100, positioned in a free space optical connection to the ultraviolet laser 101, reflects and directs 1005 the generated ultraviolet laser beam 102 to the air outlet channel 105. The second reflectors 106 of the disinfection system 100 positioned in a free space optical connection to the first reflector 103, reflect 1006 the reflected ultraviolet laser beam 102 within and throughout the length of the air outlet channel 105 in multiple directions, with the reflected ultraviolet laser beam 102 forming an ultraviolet laser beam tunnel to increase contact, utilization, and interaction of the reflected ultraviolet laser beam 102 with the exhaled air flowing in the air outlet channel 105 for irradiating and disinfecting the exhaled air.

The air supply components 801a, 801b, and 801c of the airflow system 201 blow 1007 the irradiated and disinfected air received via the air outlet channel 105 of the airflow system 201 into the enclosed spaces 803a, 803b, and 803c respectively, for inhalation by the occupants in the enclosed spaces 803a, 803b, and 803c. The guide members 806a, 806b, 806c, etc., allow 1008 only a vertical flow of the air within the enclosed spaces 803a, 803b, and 803c from the ceiling 804 to the floor 805 of the enclosed spaces 803a, 803b, and 803c such that the irradiated and disinfected air blown by the air supply components 801a, 801b, and 801c is guided to the occupants in the enclosed spaces 803a, 803b, and 803c respectively, for inhalation by the occupants in the enclosed spaces 803a, 803b, and 803c, and the air exhaled by the occupants of the enclosed spaces 803a, 803b, and 803c is guided into the respective air exhaust components 802a, 802b, and 802c for exhaustion into the air inlet channel 107, thereby minimizing and/or precluding cross-contamination of the air in the enclosed space 803. In an embodiment, the disinfection system 100 is positioned proximal to and in communication with the air inlet channel 107 for irradiating and disinfecting the exhaled air flowing through the air inlet channel 107 prior to conveyance of the air from the airflow system 201 to the air outlet channel 105. In another embodiment, the disinfection system 100 is positioned proximal to and in communication with the air inlet channel 107 and the air outlet channel 105 for irradiating and disinfecting the exhaled air flowing through the air inlet channel 107 and the air outlet channel 105 for delivery into the enclosed spaces 803a, 803b, and 803c via the air supply components 801a, 801b, and 801c respectively. The ultraviolet laser 101 of the disinfection system 100 illuminates and disinfects the air flowing the air inlet channel 107 and/or the air outlet channel 105 of the airflow system 201. The method for disinfecting air and for minimizing and/or precluding cross-contamination of the air is also implemented in other enclosed spaces, for example, 903 exemplarily illustrated in FIGS. 9A-9C, in transport vehicles such as buses, subway trains, etc., as disclosed in the descriptions of FIGS. 9A-9C.

The system 800 exemplarily illustrated in FIG. 8 minimizes and/or precludes cross-contamination of air and cross-infection between occupants in a local environment or an enclosed space 803, for example, a room in a restaurant, a hospital, a classroom, an office building, etc. The system 800 produces top-to-down ultraviolet-C (UVC) laser-disinfected airflow with a large volume, airflow system 201, for example, from the air supply components 801a, 801b, and 801c at the ceiling 804 of the enclosed space 803 towards the air exhaust components 802a, 802b, and 802c on the floor 805 of the enclosed space 803 for minimizing and/or precluding cross-contamination of the air in the enclosed space 803. In this airflow system configuration, the air supply components 801a, 801b, and 801c and the air exhaust components 802a, 802b, and 802c of the airflow system 201 are positioned at different levels, for example, at the ceiling 804 of the enclosed space 803 and on the floor 805 of the enclosed space 803 such that the air exhaled by the occupants in the enclosed spaces 803a, 803b, and 803c is directly transferred to an external environment, thereby resulting in other occupants of the surrounding enclosed spaces 803a, 803b, and 803c inhaling only disinfected air. The system 800 disclosed herein provides air that is UVC-disinfected at the output components of the airflow system 201, that is, at the air supply components 801a, 801b, and 801c of the airflow system 201, to the occupants in the enclosed spaces 803a, 803b, and 803c respectively. The system 800 disclosed herein, therefore, ensures that the occupants in the enclosed spaces 803a, 803b, and 803c only inhale disinfected air from the air supply components 801a, 801b, and 801c of the airflow system 201 respectively, while the air exhaled by the occupants in the enclosed spaces 803a, 803b, and 803c is transferred to the input components of the airflow system 201, that is, to the air exhaust components 802a, 802b, and 802c of the airflow system 201 respectively.

In comparison to ultraviolet-C (UVC) lamps and UVC light emitting diodes (LEDs), the ultraviolet laser 101, in a pulse output mode or a continuous output mode, generates a high power, ultraviolet laser beam 102 with an output power of, for example, more than several watts and in embodiments, tens, and/or hundreds, and/or thousands of watts, for optimally disinfecting the air in high volume, airflow systems in a short time. Since the ultraviolet laser 101 has more than about 100 times to about one million times higher peak power than that of UVC lamps, the ultraviolet laser 101 disinfects the air in a shorter time. The average output power of the ultraviolet laser beam 102 ranges from few watts, to several hundred watts and thousand watts in an embodiment. For example, the average output power of the ultraviolet laser beam 102 is, for example, from about 10 milliwatts (mW) to about 100 watts, and in an embodiment, about 1000 watts. The ultraviolet laser 101 disclosed herein generates, for example, about 100 times, and in an embodiment ten (10) thousand times higher output power than UVC lamps or UVC LEDs.

The disinfection system 100 uses a pulsed ultraviolet laser 101 with a pulse width of femtoseconds, picoseconds, nanoseconds, microseconds, or milliseconds, to generate a high power, ultraviolet laser beam 102 with a peak power of more than about 100 times to about tens of million times higher than that of UVC lamps and UVC LEDs to optimally inactivate and/or kill airborne pathogens and disinfect air in a short time. Furthermore, the reflection of the high power, ultraviolet laser beam 102 multiple times in multiple directions in the airflow channel(s) 105 and/or 107 provides maximum direct exposure of the air flowing through the airflow channel(s) 105 and/or 107 to the multi-reflecting high power, ultraviolet laser beam 102, thereby allowing the air to receive a substantial dosage of the ultraviolet radiation, for example, the UVC radiation, to facilitate an irreversible chemical reaction to the deoxyribonucleic acid (DNA) of the pathogens in the airflow through the airflow channel(s) 105 and/or 107 and disinfection of the airflow. This reflection of the high power, ultraviolet laser beam 102 multiple times in multiple directions in the airflow channel(s) 105 and/or 107 allows the high power, ultraviolet laser beam 102 to interact with more volume of air flowing through the airflow channel(s) 105 and/or 107 and therefore, inactivate and/or kill the airborne pathogens. Multiple second reflectors 106 provided inside the airflow channel(s) 105 and/or 107 of the airflow system 201 allow more interaction of the air flowing therethrough with the reflected high power, ultraviolet laser beam 102 therewithin, thereby allowing the air in the airflow channel(s) 105 and/or 107 to fully contact the ultraviolet light energy.

Experiments have shown that far-ultraviolet-C (UVC) light with a wavelength range of 150 nm to 300 nm kills airborne pathogens, for example, the influenza virus, human coronaviruses alpha HCoV-229E and beta HCoV-0C43, etc., potentially without harm to exposed human tissues. Low doses of about 1.7 and about 1.2 millijoule (mJ)/cm² of far-UVC light provided by the ultraviolet laser 101 inactivate about 99.9% of aerosolized coronaviruses HCoV-229E and HCoV-0C43 respectively. As all human coronaviruses have similar genomic sizes, far-UVC light is expected to show similar inactivation efficiency against other human coronaviruses including SARS-CoV-2. In an example, continuous exposure of the air flowing through the airflow channel(s) 105 and/or 107 of the airflow system 201 in a far-UVC laser beam tunnel formed in the airflow channel(s) 105 and/or 107 of the airflow system 201 by the reflectors 103, 106, etc., of the disinfection system 100 in a free space optical connection to the ultraviolet laser 101, at an exposure limit of, for example, about 3 mJ/cm²/hour, results in about 90% inactivation of the viruses in about 8 minutes, about 95% inactivation of the viruses in about 11 minutes, about 99% inactivation of the viruses in about 16 minutes, and about 99.9% inactivation of the viruses in about 25 minutes.

The foregoing examples and illustrative implementations of various embodiments have been provided merely for explanation and are in no way to be construed as limiting of the embodiments disclosed herein. Dimensions of various parts of the system disclosed above are exemplary, and are not limiting of the scope of the embodiments herein. While the embodiments have been described with reference to various illustrative implementations, drawings, and techniques, it is understood that the words, which have been used herein, are words of description and illustration, rather than words of limitation. Furthermore, although the embodiments have been described herein with reference to particular means, materials, techniques, and implementations, the embodiments herein are not intended to be limited to the particulars disclosed herein; rather, the embodiments extend to all functionally equivalent structures, methods and uses, such as are within the scope of the appended claims. It will be understood by those skilled in the art, having the benefit of the teachings of this specification, that the embodiments disclosed herein are capable of modifications and other embodiments may be effected and changes may be made thereto, without departing from the scope and spirit of the embodiments disclosed herein.

I claim:

1. A system for disinfecting air in an airflow system, the system comprising:
one or more ultraviolet lasers configured to generate and direct a high power, ultraviolet laser beam into the airflow system for inactivating and killing pathogens contained in the air flowing through the airflow system;
one or more first reflectors positioned in a free space optical connection to the one or more ultraviolet lasers, wherein the one or more first reflectors are configured and positioned to reflect and direct the ultraviolet laser beam into one or more airflow channels of the airflow system; and
one or more second reflectors positioned in a free space optical connection to the one or more first reflectors, wherein the one or more second reflectors are attached to internal surfaces of the one or more airflow channels of the airflow system, and wherein the one or more second reflectors are configured to reflect the reflected ultraviolet laser beam within and throughout length of the one or more airflow channels in a plurality of directions, with the reflected ultraviolet laser beam forming an ultraviolet laser beam tunnel to increase contact, utilization, and interaction of the reflected ultraviolet laser beam with the air flowing in the one or more airflow channels of the airflow system for irradiating and disinfecting the air.

2. The system of claim 1, wherein wavelength of pulses of the high power, ultraviolet laser beam generated by the one or more ultraviolet lasers is in a range from about 150 nanometers to about 300 nanometers.

3. The system of claim 1, wherein a pulse duration of the high power, ultraviolet laser beam generated by the one or more ultraviolet lasers is in one of milliseconds, microseconds, nanoseconds, picoseconds, and femtoseconds.

4. The system of claim 1, wherein wavelength of a continuous wave of the high power, ultraviolet laser beam generated by the one or more ultraviolet lasers is in a range from about 150 nanometers to about 300 nanometers.

5. The system of claim 1, further comprising a light transmission window positioned at a predetermined location on the one or more airflow channels of the airflow system, wherein the light transmission window is configured to pass and direct the high power, ultraviolet laser beam generated by the one or more ultraviolet lasers and reflected by the one or more first reflectors to the one or more second reflectors in the one or more airflow channels of the airflow system.

6. The system of claim 1, further comprising one or more third reflectors positioned in a free space optical connection to the one or more first reflectors and the one or more second reflectors, wherein the one or more third reflectors are operably coupled to one or more of the internal surfaces of the one or more airflow channels of the airflow system and optically aligned with the one or more second reflectors in the one or more airflow channels of the airflow system, and wherein the one or more third reflectors are configured to reflect and direct the high power, ultraviolet laser beam reflected by the one or more first reflectors to the one or more second reflectors to further increase the contact, the utilization, and the interaction of the reflected ultraviolet laser beam with the air flowing in the one or more airflow channels of the airflow system.

7. The system of claim 1, wherein the one or more airflow channels of the airflow system comprise an air inlet channel configured to allow entry of the air into the airflow system; and an air outlet channel configured to exhaust the air from the airflow system.

8. The system of claim 7, further comprising:
a plurality of air supply components operably coupled to the air outlet channel extending above a ceiling of an enclosed space, wherein the air supply components are configured to receive and blow the irradiated and disinfected air from the air outlet channel into the enclosed space; and
a plurality of air exhaust components operably coupled to the air inlet channel extending below a floor of the enclosed space, wherein the air exhaust components are configured to receive air exhaled by occupants in the enclosed space and exhaust the exhaled air into the air inlet channel for subsequent irradiation and disinfection by the high power, ultraviolet laser beam generated by the one or more ultraviolet lasers positioned proximal to one or both of the air inlet channel and the air outlet channel of the airflow system.

9. The system of claim 1, wherein each of the one or more ultraviolet lasers is one of a pulsed ultraviolet laser and a continuous wave ultraviolet laser.

10. The system of claim 1, wherein the airflow system is one of an air conditioning system, an air exchange system, an air purifier system, and an air circulation system.

11. A method for disinfecting air in an airflow system, the method comprising:
assembling a disinfection system comprising one or more ultraviolet lasers, one or more first reflectors, and one or more second reflectors, wherein the one or more first reflectors are positioned in a free space optical connection to the one or more ultraviolet lasers, and wherein the one or more second reflectors are positioned in a free space optical connection to the one or more first reflectors, and wherein the one or more second reflectors are attached to internal surfaces of one or more airflow channels of the airflow system;
generating a high power, ultraviolet laser beam by the one or more ultraviolet lasers for one of inactivating and killing pathogens contained in the air flowing through the airflow system;
reflecting and directing the generated ultraviolet laser beam by the one or more first reflectors into the one or more airflow channels of the airflow system; and
reflecting, by the one or more second reflectors, the reflected ultraviolet laser beam within and throughout length of the one or more airflow channels in a plurality of directions, with the reflected ultraviolet laser beam forming an ultraviolet laser beam tunnel to increase contact, utilization, and interaction of the reflected ultraviolet laser beam with the air flowing in the one or more airflow channels of the airflow system for irradiating and disinfecting the air.

12. The method of claim 11, wherein wavelength of pulses of the high power, ultraviolet laser beam generated by the one or more ultraviolet lasers is in a range of about 150 nanometers to about 300 nanometers.

13. The method of claim 11, wherein a pulse duration of the high power, ultraviolet laser beam generated by the one or more ultraviolet lasers is in one of milliseconds, microseconds, nanoseconds, picoseconds, and femtoseconds.

14. The method of claim 11, wherein the disinfection system further comprises a light transmission window positioned at a predetermined location on the one or more airflow channels of the airflow system, wherein the light transmission window is configured to pass and direct the high power, ultraviolet laser beam generated by the one or more ultraviolet lasers and reflected by the one or more first reflectors to the one or more second reflectors in the one or more airflow channels of the airflow system.

15. The method of claim 11, wherein the disinfection system further comprises one or more third reflectors positioned in a free space optical connection to the one or more first reflectors and the one or more second reflectors, wherein the one or more third reflectors are operably coupled to one or more of the internal surfaces of the one or more airflow channels of the airflow system and optically aligned with the one or more second reflectors in the one or more airflow channels of the airflow system, and wherein the one or more third reflectors are configured to reflect and direct the high power, ultraviolet laser beam reflected by the one or more first reflectors to the one or more second reflectors to further increase the contact, the utilization, and the interaction of the reflected ultraviolet laser beam with the air flowing in the one or more airflow channels of the airflow system.

16. A system for disinfecting air and precluding cross-contamination of the air, the system comprising:
an airflow system configured to allow a flow of air through airflow channels, wherein the airflow channels comprise an air inlet channel extending below a floor of an enclosed space, and an air outlet channel extending above a ceiling of the enclosed space;
a plurality of air supply components operably coupled to the air outlet channel of the airflow system and extending into the enclosed space from the ceiling of the enclosed space;
a plurality of air exhaust components operably coupled to the air inlet channel and positioned inside the enclosed space on the floor of the enclosed space, wherein the air exhaust components are configured to receive air exhaled by occupants in the enclosed space and exhaust the exhaled air into the air inlet channel of the airflow system; and
a disinfection system in operable communication with one or more of the airflow channels of the airflow system, the disinfection system comprising:
one or more ultraviolet lasers configured to generate and direct a high power, ultraviolet laser beam into the one or more of the airflow channels of the airflow system for one of inactivating and killing pathogens contained in the air flowing through the one or more of the airflow channels of the airflow system;
one or more first reflectors positioned in a free space optical connection to the one or more ultraviolet lasers, wherein the one or more first reflectors are configured and positioned to reflect and direct the ultraviolet laser beam to the air flowing through the one or more of the airflow channels of the airflow system; and
one or more second reflectors positioned in a free space optical connection to the one or more first reflectors, wherein the one or more second reflectors are attached to internal surfaces of the one or more of the airflow channels of the airflow system, and wherein the one or more second reflectors are configured to reflect the reflected ultraviolet laser beam within and throughout length of the one or more of the airflow channels in a plurality of directions, with the reflected ultraviolet laser beam forming an ultraviolet laser beam tunnel to increase contact, utilization, and interaction of the reflected ultraviolet laser beam with the air flowing in the one or more of the airflow channels of the airflow system for irradiating and disinfecting the air, and wherein the air supply components blow the irradiated and disinfected air from the air outlet channel of the airflow system into the enclosed space for inhalation by the occupants in the enclosed space, while the air exhaust components receive and exhaust the air exhaled by the occupants in the enclosed space into the air inlet channel of the airflow system for subsequent irradiation and disinfection by the high power, ultraviolet laser beam generated by the one or more ultraviolet lasers positioned proximal to one or both of the air inlet channel and the air outlet channel of the airflow system, thereby precluding cross-contamination of the air in the enclosed space.

17. The system of claim 16, wherein wavelength of pulses of the high power, ultraviolet laser beam generated by the one or more ultraviolet lasers is in a range from about 150 nanometers to about 300 nanometers.

18. The system of claim 16, wherein a pulse duration of the high power, ultraviolet laser beam generated by the one or more ultraviolet lasers is in one of milliseconds, microseconds, nanoseconds, picoseconds, and femtoseconds.

19. The system of claim 16, wherein the disinfection system further comprises a light transmission window positioned at a predetermined location on the one or more of the airflow channels of the airflow system, wherein the light transmission window is configured to pass and direct the high power, ultraviolet laser beam generated by the one or more ultraviolet lasers and reflected by the one or more first reflectors to the one or more second reflectors in the one or more of the airflow channels of the airflow system.

20. The system of claim 16, wherein the disinfection system further comprises one or more third reflectors positioned in a free space optical connection to the one or more first reflectors and the one or more second reflectors, wherein the one or more third reflectors are operably coupled to one or more of the internal surfaces of the one or more of the airflow channels of the airflow system and optically aligned with the one or more second reflectors in the one or more of the airflow channels of the airflow system, and wherein the one or more third reflectors are configured to reflect and direct the high power, ultraviolet laser beam reflected by the one or more first reflectors to the one or more second reflectors to further increase the contact, the utilization, and the interaction of the reflected ultraviolet laser beam with the air flowing in the one or more of the airflow channels of the airflow system.

21. The system of claim 16, further comprising guide members extending upwardly into the enclosed space from the floor of the enclosed space and positioned between each of the air supply components and a corresponding one of the air exhaust components, wherein the guide members are configured to allow only a vertical flow of the air within the enclosed space from the ceiling to the floor of the enclosed space, and wherein the guide members are configured to guide the irradiated and disinfected air blown by the air supply components to the occupants in the enclosed space for inhalation by the occupants in the enclosed space and to guide the air exhaled by the occupants in the enclosed space into the air exhaust components, thereby precluding cross-contamination of the air in the enclosed space.

22. A method for disinfecting air and precluding cross-contamination of the air, the method comprising:
assembling an airflow system comprising:
airflow channels configured to communicate a flow of air, wherein the airflow channels comprise an air inlet channel extending below a floor of an enclosed space, and an air outlet channel extending above a ceiling of the enclosed space;
a plurality of air supply components operably coupled to the air outlet channel of the airflow system and extending into the enclosed space from the ceiling of the enclosed space; and
a plurality of air exhaust components operably coupled to the air inlet channel and positioned inside the enclosed space on the floor of the enclosed space;
assembling a disinfection system comprising one or more ultraviolet lasers, one or more first reflectors, and one or more second reflectors, wherein the one or more first reflectors are positioned in a free space optical connection to the one or more ultraviolet lasers, and wherein the one or more second reflectors are positioned in a free space optical connection to the one or more first reflectors, and wherein the one or more second reflectors are attached to internal surfaces of one or more of the airflow channels of the airflow system;
receiving air exhaled by occupants in the enclosed space by the air exhaust components and exhausting the exhaled air into the air inlet channel of the airflow system;
generating a high power, ultraviolet laser beam by the one or more ultraviolet lasers of the disinfection system for one of inactivating and killing pathogens contained in the exhaled air;
reflecting and directing the generated ultraviolet laser beam by the one or more first reflectors of the disinfection system to the one or more of the airflow channels of the airflow system;
reflecting, by the one or more second reflectors of the disinfection system, the reflected ultraviolet laser beam within and throughout length of the one or more of the airflow channels in a plurality of directions, with the reflected ultraviolet laser beam forming an ultraviolet laser beam tunnel to increase contact, utilization, and interaction of the reflected ultraviolet laser beam with the exhaled air flowing in the one or more of the airflow channels of the airflow system for irradiating and disinfecting the exhaled air; and
blowing the irradiated and disinfected air from the air outlet channel of the airflow system by the air supply components into the enclosed space for inhalation by the occupants in the enclosed space, while the air exhaust components exhaust the air exhaled by the occupants in the enclosed space into the air inlet channel of the airflow system for subsequent irradiation and disinfection by the high power, ultraviolet laser beam generated by the one or more ultraviolet lasers positioned proximal to one or both of the air inlet channel and the air outlet channel of the airflow system, thereby precluding cross-contamination of the air in the enclosed space.

23. The method of claim 22, wherein wavelength of pulses of the high power, ultraviolet laser beam generated by the one or more ultraviolet lasers is in a range from about 150 nanometers to about 300 nanometers.

24. The method of claim 22, wherein a pulse duration of the high power, ultraviolet laser beam generated by the one or more ultraviolet lasers is in one of milliseconds, microseconds, nanoseconds, picoseconds, and femtoseconds.

25. The method of claim 22, wherein the disinfection system further comprises a light transmission window positioned at a predetermined location on the one or more of the airflow channels of the airflow system, wherein the light transmission window is configured to pass and direct the high power, ultraviolet laser beam generated by the one or more ultraviolet lasers and reflected by the one or more first reflectors to the one or more second reflectors in the one or more of the airflow channels of the airflow system.

26. The method of claim 22, wherein the disinfection system further comprises one or more third reflectors positioned in a free space optical connection to the one or more first reflectors and the one or more second reflectors, wherein the one or more third reflectors are operably coupled to one or more of the internal surfaces of the one or more of the airflow channels of the airflow system and optically aligned with the one or more second reflectors in the one or more of the airflow channels of the airflow system, and wherein the one or more third reflectors are configured to reflect and direct the high power, ultraviolet laser beam reflected by the one or more first reflectors to the one or more second reflectors to further increase the contact, the utilization, and the interaction of the reflected ultraviolet laser beam with the air flowing in the one or more of the airflow channels of the airflow system.

27. The method of claim 22, wherein the airflow system further comprises guide members extending upwardly into the enclosed space from the floor of the enclosed space and positioned between each of the air supply components and a corresponding one of the air exhaust components, wherein the guide members are configured to allow only a vertical flow of the air within the enclosed space from the ceiling to the floor of the enclosed space, and wherein the guide members are configured to guide the irradiated and disinfected air blown by the air supply components to the occupants in the enclosed space for inhalation by the occupants in the enclosed space and to guide the air exhaled by the occupants in the enclosed space into the air exhaust components, thereby precluding cross-contamination of the air in the enclosed space.
